(19) United States
(12) Patent Application Publication
(10) Pub. No.: US 2004/0211730 A1
(43) Pub. Date: Oct. 28, 2004

(54) Methods and compounds for controlling the morphology and shrinkage of silica derived from polyol-modified silanes

(76) Inventors: Zheng Zhang, Hamilton (CA); Yang Chen, Hamilton (CA); John D. Brennan, Dundas (CA); Michael A. Brook, Ancaster (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/647,174

(22) Filed: Aug. 25, 2003

Related U.S. Application Data
(60) Provisional application No. 60/405,308, filed on Aug. 23, 2002.
(60) Provisional application No. 60/405,309, filed on Aug. 23, 2002.

(51) Int. Cl. B01D 15/08

(52) U.S. Cl. 210/656; 210/198.2

(57) ABSTRACT

Siliceous materials are prepared by adding one or more additives, including water soluble polymers, and derivatives thereof, to sols containing tetraalkoxysilanes derived from polyols. The polymers facilitate phase separation of the growing silica gel matrix, leading to high surface area self-supporting silica gels with cure occurring at ambient temperatures. The materials also show a significant reduction in shrinkage properties.

### FIELD OF THE INVENTION

The present invention relates to methods of preparing biomolecule compatible siliceous materials, to the siliceous materials prepared using these methods and to uses of the siliceous materials, in particular as chromatographic supports, biosensors and/or to immobilize enzymes.

### BACKGROUND OF THE INVENTION

(a) Utilization of Silica as Chromatographic Support

Silica in a variety of particulate forms has been extensively utilized as chromatographic support. Partition of dissolved molecules between the hydrophilic siliceous surface and a flowing solvent permits the separation of compounds on many different scales (ng→kg scales). The efficiency of separation in these systems is related to the surface area of the silica to which the compound mixture is exposed.

The configuration of common separation systems utilizes a cylindrical bed of particulate silica in a glass, metal or polymeric cladding. A traditional approach to improving separation efficiency (theoretical plates) with such systems is to utilize longer columns of particulate silica of a given particle size (or range of sizes). Alternatively, higher separation efficiency is associated with the use of very small particles with larger surface areas.

There is an important physical limitation to practical separation with packed particulate systems. As the number of theoretical plates increases there is an attendant increase in backpressure on the column. There is, therefore, a trade off between higher separation efficiency and practical operating pressures. High pressures have attendant danger, and/or are impractical from the perspective of cost. Even with highly efficient columns operating at high pressures, the throughput that can be realized is often relatively low.¹

Significant improvement in the surface area/back pressure relationship can be realized by the use of self-supporting monolithic silica columns.^{1,2} For example, styrene monoliths have been reported to be useful for polynucleotide separation.³ The group of Tanaka, in particular, have reported the preparation of silica monoliths.⁴ Merck currently sells monolithic silica columns under the Chromolith™ label.⁵ The structure of these monoliths involves a series of distorted silica spheres fused by a layer of silica. The presence of macropores, between linked silica beads of a few microns diameter, can be clearly seen by micrographic analysis and may be more carefully established by other techniques. In addition to macropores, the silica beads typically possess a mesoporous structure (in the case of the Merck columns, a total porosity of 80% is claimed, which is made up of macro- and mesopores, the latter of which are on the order of 13 nm in diameter).⁵

(b) Problems with Existing Monolithic Silica

Silica produced by a sol-gel process is prone to shrinkage. Gelation is initiated in the presence of large quantities of solvent and, frequently, other dopants (see below). Evaporation of the solvent is accompanied by significant shrinkage forces: Si(OEt)₄-derived gels can shrink in air up to 85%.⁶ This can be problematic in a number of ways when the resulting silica is used as a chromatographic support. First, in extreme conditions, the column can fracture leading to changes/degradation in separation performance. Second, the monolith can pull away from the cladding material, providing an alternative elution pathway for the compounds to be separated. This complicates, at best, the separation. In the worst instance, the eluting mixture will bypass most of the column surface area resulting in no separation.

Several strategies have been developed to reduce the problem of shrinking. For example, use of a “drying agent” in the original sol, such as DMF, helps in the silica annealing process.⁷ The most common means to deal with shrinking is to accept that it will occur and to thermally cure the silica, essentially to completion. Hydrothermal treatment can be used to dissolve/reprecipitate the silica, which reduces the cracking that is frequently observed upon shrinking.^{8,9} Dopants like urea in the sol have been reported to facilitate the dissolution/reprecipitation process.¹⁰ An alternative strategy is to heat shrink the column cladding after shrinkage has occurred to reform an effective interface between monolith and cladding material. Finally, soluble polymers such as poly(ethylene oxide) may be added to the sol. These have the effect of increasing porosity of the monolith.¹¹

The use of sol-gel techniques provides an exceptional degree of morphological control in the preparation of silica. Thus, total porosity, pore size and shape, regularity of pore distribution, etc. can be manipulated using a variety of starting materials, reaction conditions and dopants.¹² Many of these conditions, however, are incompatible with the incorporation of fragile compounds such as biomolecules, proteins in particular. Either the synthetic conditions are damaging to protein structure (e.g., pH conditions, the presence of denaturants such as ethanol) or the final curing conditions require elevated temperatures. It is of interest to incorporate such biomolecules into silica to create materials that serve as biosensors, immobilized enzymes or as affinity chromatography supports.

(c) Applications of Monolithic Silicas to Bioaffinity Chromatogrpahy

Bioaffinity chromatography has been used widely for sample purification and cleanup,¹³ chiral separations,¹⁴ on-line proteolytic digestion of proteins,¹⁵ development of supported biocatalysts,¹⁶ and more recently for screening of compound libraries via the frontal affinity chromatography method.^{17,18} In all cases, the predominant method used to prepare protein-loaded columns has been based on covalent or affinity coupling of proteins to silica beads. However, coupling of proteins to beads has several limitations, including; loss of activity upon coupling (due to poor control over protein orientation and conformation), low surface area, potentially high backpressure (which may alter K_{d} values¹⁹), difficulty in loading of beads into narrow bore columns, difficulty in miniaturizing to very narrow columns (<50 μm i.d.), and poor versatility, particularly when membrane-bound proteins are used.¹⁸

In recent years it has been shown that a very mild and biocompatible sol-gel processing method can be used to entrap active proteins within a porous, inorganic silicate matrix.²⁰ In this method, a two-step processing method is used wherein a buffered solution containing the protein is added to the hydrolyzed silica sol to initiate gelation under conditions that are protein-compatible.²¹ Numerous reports have appeared describing both fundamental aspects of entrapped proteins, such as their conformation,^{22,23,24} dynamics,^{25,26,27} accessibility,^{24,28} reaction kinetics,^{22,29} activity,³⁰ and stability,³¹ and their many applications for catalysis and biosensing.^{20,21} A number of reports also exist describing sol-gel based immunoaffmity columns,³² and enzyme-based columns³³ although in all cases these were formed by crushing protein-doped silica monoliths and then loading the bioglass into a column as a slurry.

Very recent work on the development of protein-doped monolithic sol-gel columns has appeared from the groups headed by Zusman³⁴ and Toyo'oka.³⁵ Zusman's group have developed columns using glass fibers covered with sol-gel glass as a new support for affinity chromatography. Toyo'oka's group have used capillary electrochromatography (CEC) to both prepare protein-doped sol-gel based columns and to elute compounds. These monoliths were derived solely from TEOS or TMOS using a very high water:silicon ratio, resulting in a loosely packed monolith with large pores to allow flow of eluent. While this is a significant advance, all chromatography was done using electroosmotic flow (CEC), which separates compounds on the basis of a combination of charge, mass and affinity, and is less compatible with MS detection due to the high ionic strength of the eluent. Also, these authors did not examine the interaction of potential inhibitors with entrapped proteins on-column. This is a particularly important issue given the emergence of high throughput screening (HTS) methods based on immobilized enzymes.^{17,18,36}

The present inventors have previously described the preparation of silica from a series of sugar alcohol, sugar acid or oligo- and polysaccharide-derived silanes.⁶ These starting materials offer a number of advantages over the more classically used tetraethoxy- and tetramethoxysilanes (TEOS and TMOS, respectively). Among these are mild conditions, including a greater control of pH used in the sol (ranges from 5.5-11 are possible), very low processing temperatures, process reproducibility, reduced shrinking and compatibility with the incorporation of a variety of dopants, particularly proteins. However, there remains a need to control the shrinkage of the resulting silica to avoid the evolution of cracks. Furthermore, morphological control needs to be available such that the materials can be tailored for specific applications including chromatography, biosensors, etc.

### SUMMARY OF THE INVENTION

Siliceous materials have been prepared under mild conditions, the resulting materials showing reduced shrinkage and, under certain conditions, form a monolith having a bimodal meso/macroporous structure. Such materials are useful in chromatographic applications and are especially amenable to the entrainment of biomolecules.

Specific additives have been found by the present inventors to control the morphology and to reduce the shrinkage of siliceous materials obtained from the organic polyol modified silanes previously described in their co-pending patent application S.N. PCT/CA03/00790⁶. For example, bimodal meso/macroporous monolithic silica material was obtained by combining a water soluble polymer (for example PEO and derivatives thereof) with polyol-derived silica precursors under conditions where a phase transition, or spinodal decomposition, occurred before the material gelled. The phase transition was marked by an increase in turbity of the precursor/polymer solution. Further, certain trifunctional silanes, including water soluble polymers chemically modified to contain trifunctional silanes, provided silica having a dramatic reduction in shrinkage properties. Water soluble polymers chemically modified to contain trifunctional silanes also provided bimodal meso/macroporous monolithic silica materials.

Accordingly, the present invention includes a method of preparing siliceous materials comprising combining an organic polyol silane precursor with an additive under conditions suitable for the hydrolysis and condensation of the precursor to a siliceous material, wherein the additive is selected from the group consisting of one or more water-soluble polymers and one or more trifunctional silanes of Formula I:

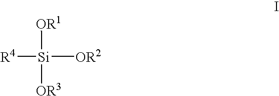

wherein R¹, R² and R³ are the same or different and represent a group that may be hydrolyzed under normal sol-gel conditions to provide Si—OH groups; and R⁴ is a group that is not hydrolyzed under normal sol-gel conditions.

The invention also includes the siliceous materials prepared using the methods of the invention as well as the use of these materials, for example, but not limited to, in chromatographic applications (particularly with macroporous materials), as bioaffinity supports, biosensors and/or for immobilizing enzymes. Further, the present invention extends to analytical and other types of hardware (for example chromatographic columns, microarrays, bioaffinity columns, etc.) comprising the materials prepared using the methods of the invention.

The mild conditions under which the siliceous materials are prepared using the methods of the present invention are compatible with proteins and other biomolecules. This allows for these types of molecules to be readily incorporated into these siliceous materials for a wide variety of applications. Also, the shrinkage of the materials prepared using the methods of the present invention is significantly reduced when compared to TEOS- or TMOS-derived materials (as well as polyol-silane derived materials which were prepared under conditions previously reported⁶), which again, provides a more stable environment for entrained biomolecules.

The present inventors have also developed biomolecule compatible, bimodal meso/macroporous silica materials using the method of the present invention. It has been shown that these materials can be used for protein entrapment and that capillary columns based on these materials can be prepared that are suitable for pressure driven liquid chromatography and compatible with MS detection.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the FT-IR spectra of gluconamide-modified, DGS-derived silica;

FIG. 2 shows FT-IR spectra of maltonamide-modified, DGS-derived silica

FIG. 3 shows solid-state ¹³C CPMAS NMR spectra: (a) 8, (b) 15;

FIG. 4 shows solid-state ²⁹Si CPMAS NMR spectra: (a) 8, (b) 15;

FIG. 5 is a bar graph showing the shrinkage data for samples 6-15 over 45 days;

FIG. 6 is a bar graph showing the mobility measurements of crushed particles from samples 6 to 11;

FIG. 7 is a graph showing the gel time of DGS doped with different molecular weight and different concentration of PEO solution. The open legend means transparent gel at gel time, and the closed legend means phase separation before gelation;

FIG. 8 shows electron micrographs of silica prepared from DGS using both PPG-NH₂ and PEO as dopants showing the change in morphology as the quantity of PPG-NH₂ was increased; i) PEO/PPG-NH₂=1000/1 ii) (b) PEO/PPG-NH₂=1000/5 and (c) PEO/PPG-NH₂=1000/10;

FIG. 9 shows electron micrographs of silica derived from DGS/PEO A: where the phase separation was not allowed to go to completion; B: after complete phase separation and gelation; C: Close-up of B;

FIG. 10 shows TGA of silica monoliths, after washing and freeze drying, derived from 1: DGS, and 2: DGS/PEO (from 0.5 g DGS/500 μL H20/500 μL of 0.05 g/ml PEO 100,000 MW solution);

FIG. 11 shows DSC of silica derived from: A: DGS; B: DGS+100000 MW PEO;

FIG. 12 shows silica gel prepared from DGS A: Using polyNIPAM as dopant; B: Using PEO with terminal Si(OEt)₃ groups as dopant (see experimental section in both cases);

FIG. 13 shows UV-visible spectra of the washing solution of FITC labelled HSA;

FIG. 14 shows Confocal Microscopy Images of the PEO/DGS gel entrapped with fluorescent FITC-HSA after the 3^{rd} washing;

FIG. 15 shows a chematic of the apparatus used for FAC/MS. A switch valve is used to switch from buffer to buffer+analyte, allowing continuous infusion of analytes onto the column. The column outlet is connected to a mixing tee for addition of makeup buffer that flows directly into the PE/Sciex API 3000 triple-quadrupole mass spectrometer.

FIG. 16 shows SEM images of a sol-gel derived column containing DGS/PEO/APTES after 5 days of aging. Panel A: image of monoliths formed in 1 mm capillaries that pulled away from the capillary wall and were removed from the capillary under flow; Panel B: magnified image of a monolith in a 250 μm capillary showing the bimodal pore distribution within the sol-gel derived monolith.

FIG. 17 shows brightfield images of a filled capillary (250 μm i.d.) after 3 months of aging in buffer (Panel A), and after 24 hours of storage in a dessicator (Panel B).

FIG. 18 shows FAC/MS data showing the effects of surface modification on non-slective adsorption. Panel A: unmodified DGS/PEO monoliths; Panel B: DGS/PEO monoliths containing 0.3% w/v DMDMS; Panel C: DGS/PEO monoliths containing 0.03% w/v poly(allylamine) MW 17,000; Panel D: DGS/PEO monoliths containing 0.3% w/v APTES. The order of elution for all chromatographs is: Methotrexate, Fluorescein, Trimethoprim, Pyrimethamine.

FIG. 19 shows typical FAC/MS traces obtained using protein-loaded and blank DGS/PEO/APTES monolithic columns. Panel A: blank columns containing no protein; Panel B: column containing 50 pmol DHFR (initial loading); Panel C: column containing an initial loading of 50 pmol of heat-denatured DHFR; Panel D: columns containing and initial loading of 50 pmol of HSA. Coumarin, pyrimethamine, trimethoprim and folic acid were infused at 20 nM. Fluorescein was infused at 100 nM. All traces are normalized to the maximum signal obtained after compound breakthrough.

FIG. 20 shows the determination of K_{d} and Bₜ values for DHFR columns based on the effect of ligand concentration on breakthrough volume. Panel A: Superimposed FAC/MS traces at 4 different ligand concentrations relative to a blank column; Panel B: Plot of V vs. [A]. Note that the data is obtained from the first run performed on four individual DHFR loaded columns and one blank column.

FIG. 21 shows column to column reproducibility for elution of 5 compounds. Panels A and B show FAC/MS data obtained for two different columns cut from one continuously filled capillary. Columns were infused with a solution containing 20 mM of coumarin, folate, trimethoprim and pyrimethamine and 100 mM Fluorescein in 2 mM ammonium acetate. The order of elution was coumarin (2.7 and 2.6 min), fluorescein (3.1 and 2.9 min), folate (8.9 and 8.4 min), trimethoprim (37.2 and 41.4 min) and pyrimethamine (56.7 and 61.4 min).

FIG. 22 shows run-to-run reproducibility and column regeneration for a 5 day old DGS/PEO/APTES column containing an initial loading of 50 pmol of DHFR. The order of elution for panel A (initial run) was coumarin, fluorescein, folate, trimethoprim and pyrimethamine. After re-equilibrating the column with 2 mM ammonium acetate, 30 mM folate was flowed through the column for 40 min to displace tightly-bound affinity analytes in panel B. The column was then re-equilibrated with 2 mM ammonium acetate before FAC was repeated in panel C.

### DETAILED DESCRIPTION OF THE INVENTION

(i) Methods of the Invention

The present inventors have developed methods to control the morphology and shrinkage characteristics of siliceous materials derived from organic polyol modified silanes. Specifically, it has been found that the addition of higher molecular weight PEO, or other water soluble polymers, to organic polyol-based sols under conditions where a phase transition, or spinodal decompostion, occurs before gelation, leads to bimodal meso/macroporous monolithic silica material. Further, it has been found that the addition of trifunctional silanes conjugated through an alkyl amide linkage to sugar lactones (including gluconamide, maltonamide and dextronamide), to organic polyol-based tetrafunctional silanes, including as representative, non-limiting examples, diglycerylsilane (DGS) and monosorbitylsilane (MSS), provides siliceous materials having a dramatic reduction in shrinkage properties. Similarly, PEO modified with a trifunctional silane through a propyl ether linkage led to a reduction in silica shrinkage. Accordingly, a route to siliceous materials that have reduced shrinkage compared to TEOS-derived gels, which are readily formed over a wide range of pHs and which may be prepared at ambient or slightly higher (e.g., 37 C) temperatures, without the necessity for heat curing or air drying, has been developed. As a result, it is possible to dope these siliceous materials with a variety of species, in particular biomolecules such as proteins.

Accordingly, the present invention relates to a method of preparing siliceous materials comprising combining an organic polyol silane precursor with an additive under conditions suitable for the hydrolysis and condensation of the precursor to a siliceous material, wherein the additive is selected from the group consisting of one or more water-soluble polymers and one or more trifunctional silanes of Formula I:

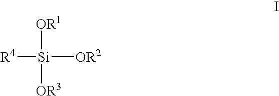

wherein R¹, R² and R³ are the same or different and represent a group that may be hydrolyzed under normal sol-gel conditions to provide Si—OH groups; and R⁴ is group that is not hydrolyzed under normal sol-gel conditions.

The water soluble polymer may be selected from any such compound and includes, but is not limited to: polyethers, for example, polyethylene oxide (PEO), polyethylene glycol (PEG), amino-terminated polyethylene glycol (PEG-NH₂); polypropylene glycol (PPG), polypropylene oxide (PPO), polypropylene glycol bis(2-amino-propyl ether) (PPG-NH₂); poly alcohols, for example, polyvinyl alcohol; polysaccharides; poly(vinyl pyridine); polyacids, for example, poly(acrylic acid); polyacrylamides e.g. poly(N-isopropylacrylamide) (polyNIPAM); and polyallylamine (PAM). Preferably the water soluble polymer is selected from PEO, PEO—NH₂, PEG, PPG-NH₂, polyNIPAM and PAM. More preferably, the water soluble polymer is selected from PEO, PEO—NH₂ and polyNIPAM. By “water soluble” it is meant that the polymer is capable of being formed into an aqueous solution having a suitable concentration. It should be noted that the terms “oxide” (as in polyethylene oxide) and “glycol” (as in polyethylene glycol) may be used interchangeably and the use of one term over the other is not meant to be limiting in any way.

In embodiments of the invention, OR¹, OR² and/or OR³ are the same or different and are derived from organic mono-, di-, or polyols. By “polyol”, it is meant that the compound has more the one alcohol group. The organic portion of the polyol may have any suitable structure ranging from straight and branched chain alkyl and alkenyl groups, to cyclic and aromatic groups. For the preparation of biomolecule compatible silicas, it is preferred for the organic polyol to be biomolecule compatible. In an embodiment of the invention, the groups OR¹, OR² and/or OR³ are derived from sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydroylze to yield such compounds. The polyol may be a monosaccharide, the simplest of the sugars or carbohydrate. The monosaccharide may be any aldo- or keto-triose, pentose, hexose or heptose, in either the open-chained or cyclic form. Examples of monosaccharides that may be used in the present invention include, but are not limited to, allose, altrose, glucose, mannose, gulose, idose, galactose, talose, ribose, arabinose, xylose, lyxose, threose, erythrose, glyceraldehydes, sorbose, fructose, dextrose, levulose and sorbitol. The polyol may also be a disaccahride, for example, but not limited to, sucrose, maltose, cellobiose and lactose. Polyols also include polysaccharides, for example, but not limited to dextran, (500-50,000 MW), amylose and pectin. Other organic polyols that may be used include, but are not limited to glycerol, propylene glycol and trimethylene glycol. In embodiments of the present invention, the group OR¹, OR² and/or OR³ is derived from a polyol selected from glycerol, sorbitol, maltose, trehalose, glucose, sucrose, amylose, pectin, lactose, fructose, dextrose and dextran and the like. In further embodiments of the present invention, the organic polyol is selected from glycerol, sorbitol, maltose and dextran.

In other embodiments of the invention, OR¹, OR² and OR³ are the same and are selected from C₁₋₄alkoxy, for example, methoxy or ethoxy, aryloxy and arylalkyleneoxy. In further embodiments of the invention, OR¹, OR² and OR³ are all ethoxy.

The term “aryloxy” as used herein means phenoxy or naphthyloxy wherein, the phenyl and naphthyl groups may be optionally substituted with 1-5 groups, preferably 1-3 groups, independently selected from the group consisting of halo (fluoro, bromo, chloro or iodo), C₁₋₆alkyl, C₁₋₆-alkoxy, OH, NH₂, N(C₁₋₆alkyl)₂, NHC₁₋₆alkyl, C(O)C₁₋₆alkyl. C(O)NH₂, C(O)NHC₁₋₆alkyl, OC(O)C₁₋₆alkyl, OC(O)OC₁₋₆alkyl, NHC(O)NHC₁₋₆alkyl, phenyl and the like.

The term “arylalkyleneoxy” as used herein means aryl-(C₁₋₄)-oxy wherein aryl has the same meaning as in “aryloxy”. Preferably, “arylalkyleneoxy” is a benzyl or naphthylmethyl group (i.e. aryl-CH₂—O).

It should be noted that the groups OR¹, OR² and OR³ are capable of participating directly in the hydrolysis/polycondensation reaction. In particular, these functional groups are alkoxy groups attached to the silicon atom at oxygen, i.e., “Si—OR”, which may be hydrolyzed to provide a “Si—O—H”, which can condense with other “Si—O—H” or “Si—OR” groups to provide “Si—O—Si” linkages and eventually a three-dimensional network within a firm gel. Trifunctional silanes form silsesquioxanes upon hydrolysis and there is a lower degree of crosslinking in systems derived therefrom, in particular when compared with systems derived from tetrafunctional silanes. The remaining group attached to the silicon atom (R⁴) is a group that generally does not participate directly in the hydrolysis/polycondensation reaction.

R⁴ is a group that is not hydrolyzed under normal sol-gel conditions and preferably is stabilizing to biological substances, in particular proteins. In specific embodiments, R⁴ is selected from one of the following groups:

polyol-(linker)-;

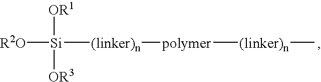

wherein n is 0-1 and OR¹, OR² and OR³ are as defined above. The term “polyol” in R⁴ has the same definition as described above for the groups OR¹, OR² and OR³. The term “polymer” in R⁴ refers to any water soluble polymer, such as, but not limited to: polyethers, for example, amino-terminated polyethylene oxide (PEO), polyethylene glycol (PEG), polyethylene glycol bis(2-amino-propyl ether) (PEG-NH₂); polypropylene glycol (PPG), polypropylene oxide (PPO), polypropylene glycol bis(2-amino-propyl ether) (PPG-NH₂); poly alcohols, for example, polyvinyl alcohol; polysaccharides; poly(vinyl pyridine); polyacids, for example, poly(acrylic acid); polyacrylamides e.g. poly(N-isopropylacrylamide) (polyNIPAM); and polyallylamine (PAM). A linker group is required (i.e. n=1) when a direct bond between the silicon atom and the polymer would be hydrolyzed under normal sol-gel conditions. In embodiments of the invention, the polymer is a water soluble polyether such as PEO.

The sugar and polymer residues may be attached to the silicon atom through any number of linkers. Such linkers may be based on, for example, alkylene groups (i.e. —(CH₂)ₘ—, m=1-20, preferably 1-10, more preferably 1-4), alkenylene groups (i.e. —(CH═CH)ₘ—, m=1-20, preferably 1-10, more preferably 1-4), organic ethers, thioethers, amines, esters, amides, urethanes, carbonates and ureas. A person skilled in the art would appreciate that they are numerable linkers that could be used to connect the group, R⁴, to the silicon atom.

By “biomolecule compatible” it is meant that a substance either stabilizes proteins and/or other biomolecules against denaturation or does not facilitate their denaturation.

The terms “biomolecule” or “biological substance” as used herein, are interchangeable and means any of a wide variety of proteins, enzymes and other sensitive biopolymers including DNA and RNA, and complex systems including whole plant, animal and microbial cells that may be entrapped in silica. The biomolecule is preferably dissolved in a suitable solvent, for example an aqueous buffer solution, such as TRIS buffer. In an embodiment of the invention, the biological substance is in its active form.

By “normal sol gel conditions” it is meant the conditions used herein to effect hydrolysis and condensation of the organic polyol derived silanes. This includes, in aqueous solution, at a pH in the range of 1-13, preferably in the range 4-11.5, and temperatures in the range of 0-80 C, and preferably in the range 0-40 C, and optionally with sonication and/or in the presence of catalysts known to those skilled in the art of room temperature vulcanization, including acids, amines, dialkyltin esters, titanates, etc.

Illustrative of compounds of Formula I of the present invention, are two classes of the trifunctional silanes based on saccharides which were prepared as described hereinbelow: monosaccharide- (compound 1) and disaccharide-(compounds 2 and 3) based trifunctional silanes are shown in Schemes 1 and 2. Also prepared were polymeric bis(trifunctional silanes) 5 (see Scheme 3).

Although in both of the saccharide examples shown in Schemes 1 and 2, many different opportunities for modification with silanes exist, it was chosen to modify the anomeric hemiacetal centre at the terminus of the saccharidic chains. Oxidation of any of the sugars converts the anomeric hemiacetal into the lactone (Scheme 1). This could then be opened by an amino-modified alkoxysilane to produce a sugar-modified coupling agent.³⁸ The key functional group tethering the two groups in this case is an alkylamide. Examples of such sugar modified silanes prepared herein are shown in Scheme 2.

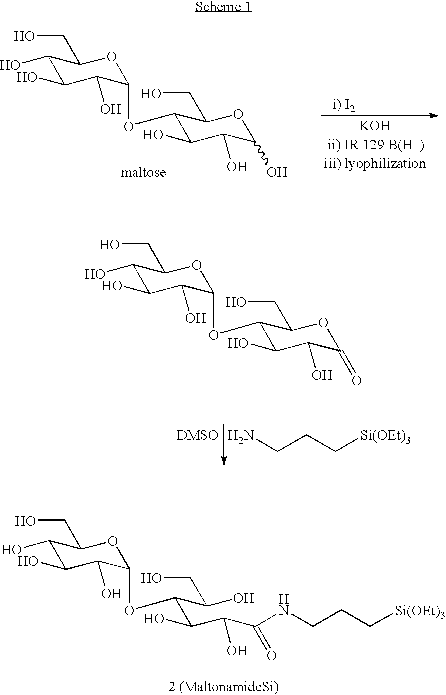

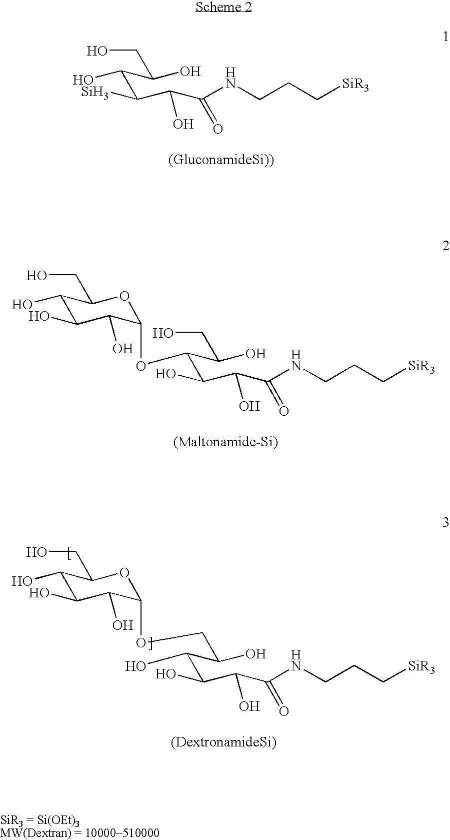

Illustrative of compounds of Formula I wherein R⁴ is

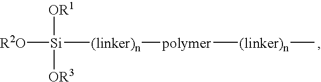

wherein OR¹, OR² and OR³ are as defined above, are compounds 5 shown in Scheme 3. Compounds 5 can be prepared, for example, by reacting poly(ethylene oxide), first with allyl bromide (or any other suitable allylating reagent), followed by reaction with a trialkoxy-, triarylalkyleneoxy- or triaryloxysilane, in the presence of a catalyst, such as a platinum-derived catalyst, as shown in Scheme 3. When modified PEO polymers are used, for example the compound of Formula 5, it is preferred that the starting PEO have a MW of greater than about 2000 g/mol. In this example the linker is an alkylene group, with m=3. Note some allyl-terminated PEO polymers 4 are commercially available. It would be apparent to one skilled in the art that other levels of functionality can also be used to bind these species to the siliceous matrix, such as: R₃₋ₖJₖSi-linker-polymer-linker-SiJₖR₃₋ₖ and polymer-linker-SiJₖR₃₋ₖ where k=1-3 and J is a group that can participate in hydrolysis and condensation with the silica network.

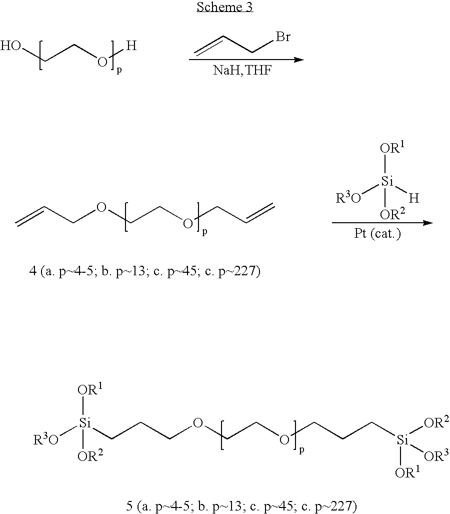

As stated above, the organic polyol derived silane precursors have been described in the inventors' co-pending patent application (PCT patent application S.N. PCT/CA03/00790)⁶, the contents of which are incorporated herein by reference. The term “polyol” once again has the same definitions as described above. Although a wide variety of ratios of sugar/silicon are readily prepared (e.g., monosorbitylsilane (MSS), disorbitylsilane, trisorbitylsilane), as will be appreciated by one skilled in the art, the cure behavior of such compounds differs widely. The resulting polyol modified silanes listed below are particularly convenient for the method of the invention: diglycerylsilane (DGS), monosorbitylsilane (MSS), monomaltosylsilane (MMS), dimaltosylsilane (DMS) and a dextran-based silane (DS). One of skill in the art can readily appreciate that other molecules including simple saccharides, oligosaccharides, and related hydroxylated compounds can also lead to viable silica precursors. Higher molecular weight polyols and polysaccarides, e.g. dextran and the like, do not completely leach from the silica, once formed.

The preparation of silica from sugar-modified silanes such as glycerol (DGS—diglycerylsilane; or MSS—monosorbitylsilane) has been previously reported.⁶ In those cases, all silicon atoms contained in the resulting gel were tetrafunctional, Q-type³⁹ (four bonds to oxygen, Si(OR)₄). Co-hydrolysis of any of the water soluble polymers or compounds of Formula I with DGS or MSS led to silica possessing very different properties.

The hydrolysis and polycondensation of the organic polyol derived silanes in the presence of one or more additives typically occurred upon standing of the reagents in aqueous solution or with sonication to assist in dissolution. In preferred embodiments of the invention, the additives are added as solutions in suitable buffers. The aqueous solution may be adjusted to a pH in the range of 4-11.5 (and may be tailored to the biomolecule, if any is to be entrained in the matrix), using a buffer, for example Tris buffer, to initiate hydrolysis and condensation. In an embodiment of the invention, the pH is adjusted so that it is in a range of about 4-10. The resulting solution will eventually gel (lose the ability to flow) and the material may be allowed to cure or age for sufficient period of time. A person skilled in the art can determine this time depending on the desired application for the siliceous material. The term “cure” or “age” means the continued evolution of the silica matrix upon aging of the silica following gelation. Once the material is sufficiently cured, it may be dried before use. The material may be molded into any desired shape, for example, films, spots, fibres, monoliths, pellets, granules, tablets, rods and bulk, as the solution becomes viscous but before it becomes completely gelled.

It has been found that when the additive is a trifunctional compound of Formula I, siliceous materials having reduced shrinkage are produced. Accordingly, in embodiments of the invention, there is provided a method of preparing siliceous materials with low shrinkage characteristics comprising:

(a) combining an aqueous solution of one or more compounds of Formula I with an aqueous solution of an organic polyol silane precursor

(b) adjusting the pH of the solution in (a) to about 4-11.5;

(c) allowing the solution of (b) to gel;

(d) aging the gel of (c); and

(e) drying the aged gel in air.

In further embodiments, the compound of Formula I is selected from those wherein R⁴ is selected from one of the following groups:

polyol-(linker)-; and

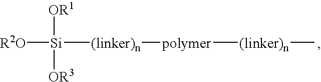

wherein n is 0-1 and OR¹, OR² and OR³ are as defined above.

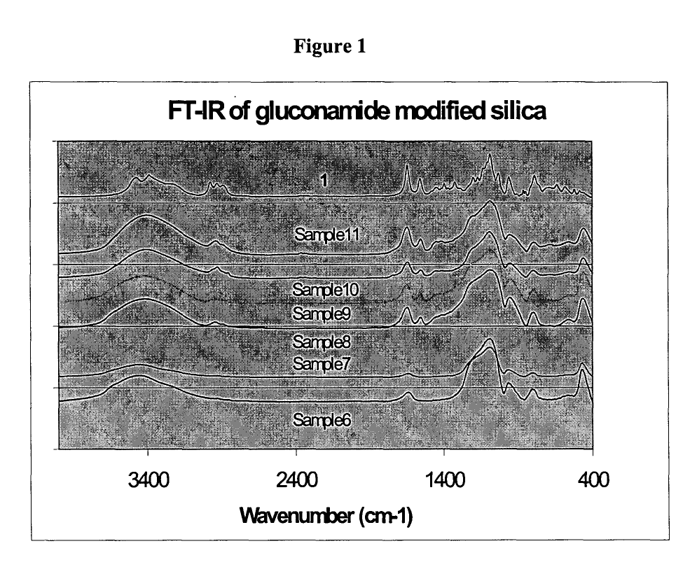

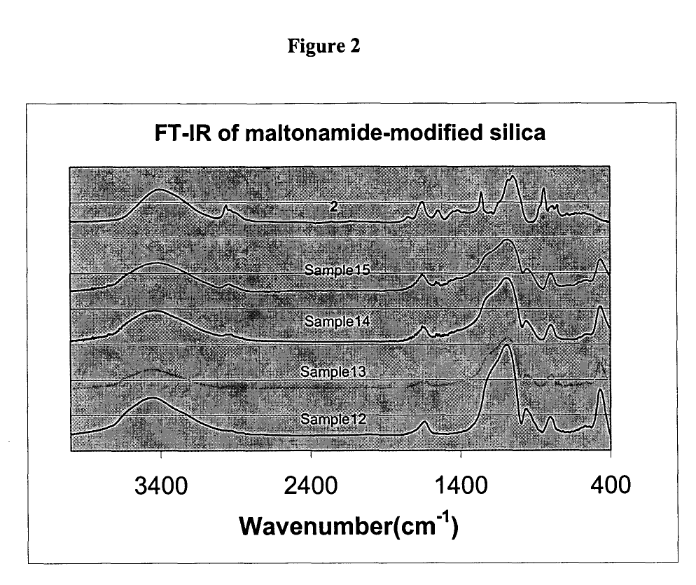

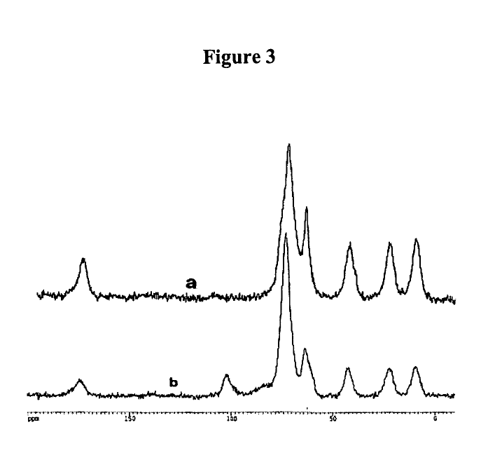

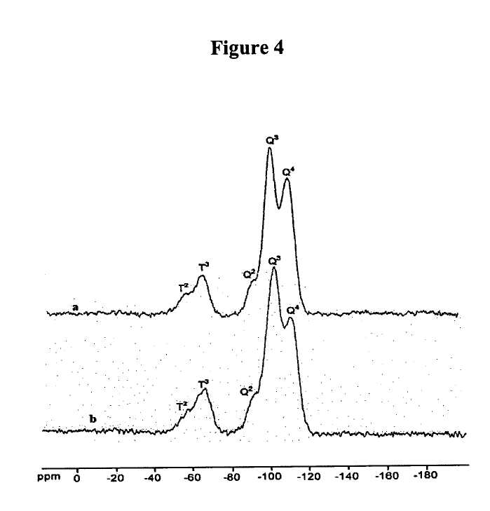

A series of compounds derived from DGS combined with gluconamide-Si(OEt)₃ 1, maltonamide-Si(OEt)₃, 2 or PEO—[Si(OEt)₃]₂, 5 respectively, were prepared. Gelation times for the composites depended upon the ratio of starting materials. In particular, there was a trend to slower curing (gelling) with an increase in the proportion of the additive (Table 1). These materials could be characterized by standard spectroscopic techniques including IR and NMR (See FIG. 1-4 and Table 2). The former, in particular, is diagnostic because of the amide linkages that appear in the region between 1650 and 1700 cm⁻¹ (FIGS. 1-2).

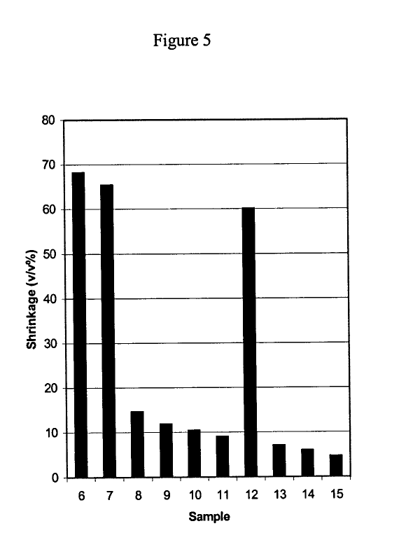

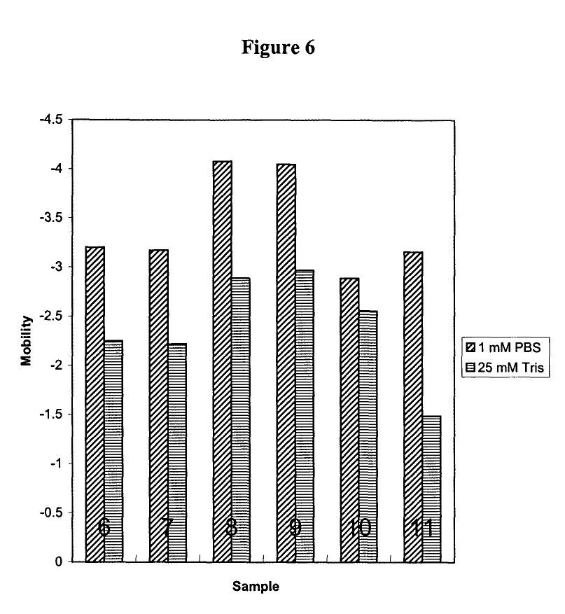

The physical behavior of the siliceous materials prepared by combining a organic polyol silane precursor with a compound of Formula I was also studied. As stated above, the most significant impact on the behavior of the resulting products can be seen in the degree of shrinkage. Normally, when allowed to rest in the open environment (i.e., not under water), shrinkage of DGS gels occurs to a level of up to approximately 66% (see sample 6, FIG. 5), much less than TEOS-derived gels which shrink approximately 85%. By contrast, incorporation of the sugar-modified trifunctional silanes dramatically reduced shrinkage over the same time period (45 days) to less than 15% (samples 8-11, 13-15, FIG. 5). Reduced shrinkage was also observed when DGS was hydrolyzed and condensed in the presence of compounds 5. Mobility measurements were undertaken in order to assess the degree to which the trifunctional silanes modified the behaviors of the resulting siliceous surfaces. These results are shown in Table 3, FIG. 6. In all cases, the surfaces remain anionic.

The hydrolysis of DGS or MSS (and related compounds) leads to silica networks contaminated with polyol. These networks shrink far less than silica prepared from TEOS and are also more protein compatible, as no denaturant is present during gel formation. In addition, the pH used for the gel synthesis can be adapted to the specific protein to be entrapped since, as stated above, gel formation conveniently occurs without supplemental catalysis over a pH range of 4-11.5. The addition of trifunctional compounds based on sugar lactones or polymers, significantly changed the behavior of the resulting cure process, and most significantly decreased shrinkage in the final material.

The present invention also includes siliceous materials prepared using the method of the invention. Accordingly, the invention relates to siliceous materials having reduced shrinkage properties. By “reduced shrinkage” properties it is meant that the siliceous material shrinks in the range of about 5-15% (v/v) over a period of 45 days at in air room temperature.

In other aspects of the present invention, bimodal meso/macroporous silica monoliths were formed when the organic polyol silane precursors were combined with one or more water soluble polymers and/or compounds of Formula I, wherein R⁴ is group selected from polymer-(linker)ₙ- and

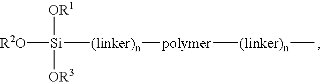

under conditions where the resulting sol undergoes a phase transition before gelation.

Accordingly, the present invention includes a method of preparing monolithic silica materials comprising combining an organic polyol silane precursor with one or more additives selected from water-soluble polymers and compounds of Formula I, wherein R⁴ is group selected from polymer-(linker)ₙ- and

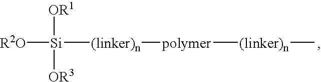

under conditions where a phase transition occurs before gelation.

In embodiments of the invention R⁴ is

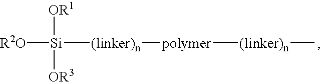

In further embodiments of the invention, the linker group is a C₁₋₄alkylene group and n is 1. The selections for OR¹, OR² and OR³ are the same as those defined above.

The present invention also extends to the novel bimodal meso/macroporous silica monoliths prepared using the method of the invention. The invention therefore relates to a silica monolith with improved shrinkage characteristics, that is compatible with biomolecules and which is prepared at ambient temperature.

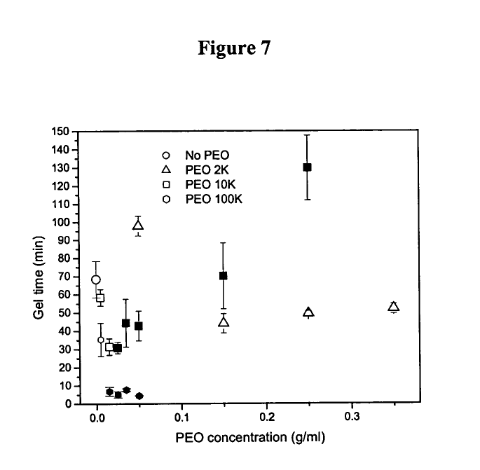

The conditions where a phase transition occurs before gelation may vary depending mainly on the identity of the water-soluble polymer (Table 4). When the water-soluble polymer is PEO, the timing of the gelation was dependent on both the PEO concentration and molecular weight (FIG. 7). In order for phase transition to occur before gelation, it is preferred that the non-functionalized PEO be of relatively high molecular weight (MW), for example greater than about, 5000, preferably greater than about 7500, more preferably greater than about 10,000 g/mol, most preferably greater than about 100,000 g/mol, and at relatively high concentration, for example greater than about 0.005 g/mL of final solution, preferably greater than about 0.025 g/mL of final solution. Macroporous silica monoliths were also formed when poly(N-isopropylacrylamide) (polyNIPAM) was used as the water soluble polymer. The molecular weight of the polyNIPAM may be greater than about, 5000, preferably greater than about 7500, more preferably greater than about 10,000 g/mol, most preferably greater than about 100,000 g/mol, and its concentration, may be greater than about 0.005 g/mL of final solution, preferably greater than about 0.025 g/mL of final solution. For amino-modified PEO (PEO—NH₂) the molecular weight may be greater than about 1000 g/mol, preferably greater than about 2000 g/mol, more preferably greater than about 3000 g/mol and its concentration, may be greater than about 0.005 g/mL of final solution, preferably greater than about 0.025 g/mL of final solution.

The effect of different functional groups on the water soluble polymer on cure characteristics was pronounced. Non-functional PEO of 10,000 MW was needed for phase separation to occur before gelation. By contrast, poly(ethylene oxide) bearing terminal amino groups (PEO—NH₂) could form macroporous structures with molecular weights of only 3400, and PEO terminated with O(CH₂)₃Si(OEt)₃ groups (PEO-TES₂) could form macroporous structure with molecular weights of greater than about 200.

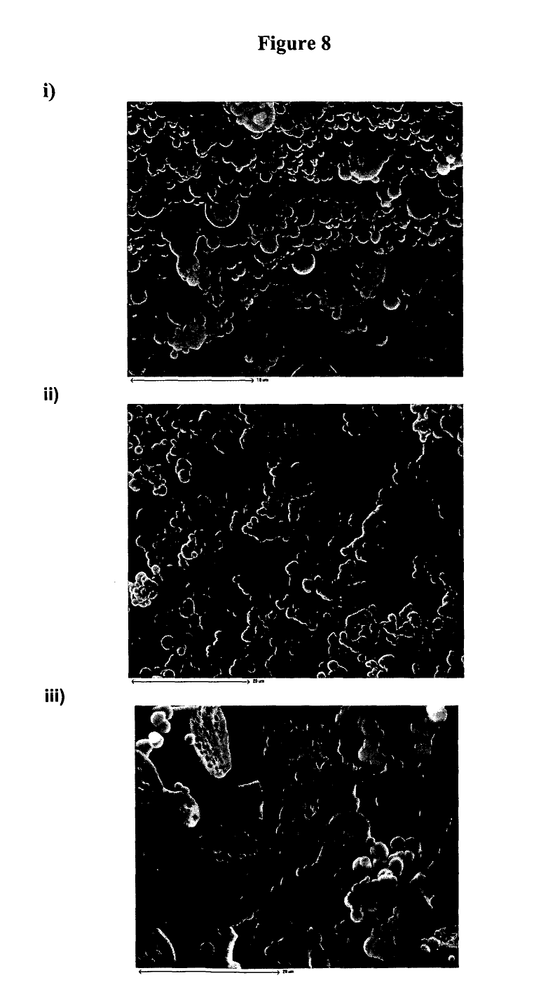

Macroporous silica monoliths could also be prepared by using a mixture of water soluble polymers. In this case, the morphology of the resulting silica was affected by the concentration, molecular weights, and character of the polymers. For example, addition of various amounts of PPG-NH₂ to a DGS-PEO sol led to silicas of very different morphology (see FIG. 8; the base recipe consists of 0.2 g DGS/200 μL H₂O+60 μL of 0.5 g/ml PEO 10,000 MW, to which was added a PPG-NH₂ solution comprised of PPG-NH₂ 200 MW 0.5 g/mL such that the final PEO/PPG-NH₂ ratios were i) 1000/1 ii) 1000/5, and iii) 1000/10).

A person skilled in the art can readily determine when a phase transition has occurred, for example, by observing the evolution of turbidity in the sol. As used herein, the time when the solution became totally opaque was recorded as the phase separation time (tₚₛ) and the time with the opaque phase lost its ability to flow was recorded as the gel time (t_{gel}).

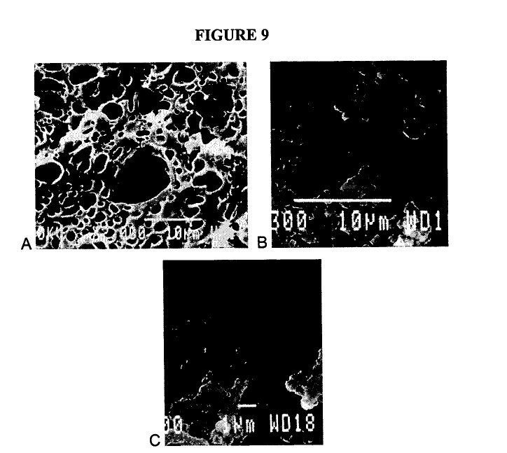

The silica formed as a result of gelation after phase separation consists of small asymmetric beads fused together to create an open structure. The way in which the open structure evolves could be seen by washing unreacted starting material or low molecular weight oligomers from the gel prior to complete reaction of the alkoxysilane. The evolution of the gel can be seen in FIG. 9. The size of the aggregates is a function of the specific recipe used, and in particular depends on the molecular weight and type and weight percent of additive incorporated.

The aggregated silica beads that comprise the monolith are mesoporous in nature. This is clearly seen from the nitrogen absorption data (Table 5) which shows average pore sizes of 3.3 nm.

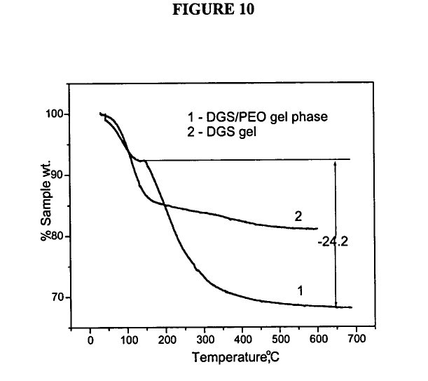

The silica macroporous monoliths formed using the method of the invention contain significant quantities of the organic polymer used to cause phase separation. Thermogravimetric analysis (TGA) showed that significantly greater quantities of organic material were found in the gels formed from DGS and doped with polymers than those which contained DGS and water in the absence of dopants (FIG. 10). Additional weight losses on heating due to evaporation of water, on the order of 10-15%, were observed in gels doped with polymer.

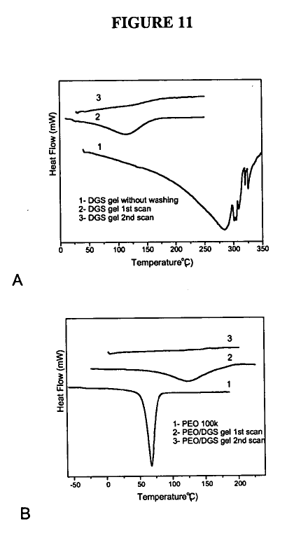

Further characterization of the nature of the sol-gel monoliths prepared using the method of the invention was available from calorimetry. Differential scanning calorimetry (DSC) of the gel resulting from reaction of DGS, water and PEO shows features associated with the glycerol (from DGS) but not with the polymeric dopant. Thus, an unwashed sample of silica derived only from DGS shows loss of glycerol above 200 C (FIG. 11A). By contrast, the washed sample shows no glycerol in the first heating cycle and no significant thermal events in the second heating cycle. The melting point of pure PEO (MW 100,000) is 67 C as seen in FIG. 11B. In the silica prepared from DGS and PEO, DSC shows no evidence of entrained domains of PEO or glycerol in the gel after crushing, washing and drying the gel. The peak in curve 2 corresponds to loss of some water (FIG. 11B). However, there is approximately 24% PEO remaining in the gel after washing (FIG. 10). Thus, the data from these gels is consistent with a silica structure containing dispersed PEO as can be seen from Table 6.

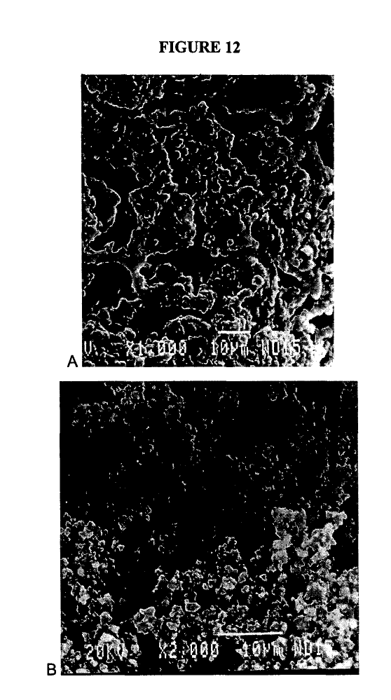

Use of polymers other than PEO can result in a different morphology in the resulting monolithic silica. These differences are readily visible in electron micrographs. A comparison of the silica prepared with PEO (FIG. 9B) with that prepared in the presence of polyNIPAM (FIG. 12A) shows very different aggregation behavior. The addition to DGS of PEO, PPG-NH₂ and compounds of Formula I, wherein R⁴ is polymer-(linker)ₙ- or

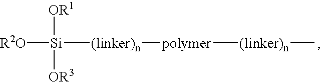

under conditions where a phase transition occurs before gelation, changed the aggregate size and morphology in a different manner (FIGS. 8 and 12B).

It was noted above that a particular advantage of the methods of the present invention is that they are amenable for the preparation of biomolecule-doped siliceous materials. Accordingly, the present invention further relates to a method of preparing siliceous materials comprising combining an organic polyol silane precursor, a biomolecule of interest and an additive under conditions suitable for the hydrolysis and condensation of the precursor to a siliceous material, wherein the additive is selected from the group consisting of one or more water-soluble polymers and one or more trifunctional silanes of Formula I:

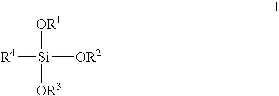

wherein R¹, R² and R³ are the same or different and represent a group that may be hydrolyzed under normal sol-gel conditions to provide a Si—OH group; and R⁴ is group that is not hydrolyzed under normal sol-gel conditions.

The present invention further relates to the siliceous material comprising a biomolecule or biological substance entrapped therein wherein the siliceous material is prepared using the methods described hereinabove.

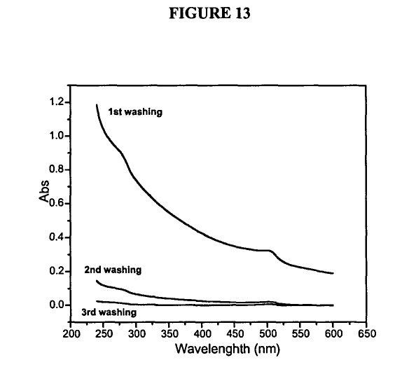

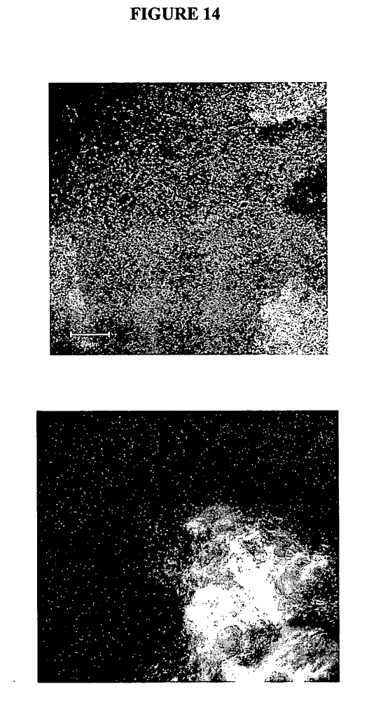

The incorporation of biomolecules into the silica monoliths prepared using the method of the present invention is exemplified by the silica formed in the presence of the surface active protein human serum albumin⁴⁰ (HSA) or lysozyme using a recipe incorporating 100,000 MW PEO as a dopant (Table 7). It was possible to partially remove the protein by extensive washing, as shown using fluorescently labelled HSA: the more PEO in the original recipe, the less protein remains in the column after washing. The washing liquors from the PEO/DGS/FITC-labelled HSA silica were examined by UV-visible spectroscopy. A very weak absorption signal could still be detected in the 3^{rd} washing (FIG. 13). However, the resulting gel still contained significant quantities of HSA, as shown by the strong fluorescent signal observed by confocal microscopy after 3 days total soaking (FIG. 14). The addition of PPG-NH₂ to the sol changes the ultimate degree of proteins retention. As shown in Tables 7 and 8, PPG-NH₂ is much more efficient than other polymers in retaining proteins. Thus, in addition to morphological changes provided by the addition of a water soluble polymer, these polymers also play a role in controlling the total protein content in the silica monolith.

The bimodal meso/macroporous monoliths prepared using the methods of the invention undergo shrinkage, as is common for sol-gel derived silica. However, the magnitude of shrinkage of these materials is also significantly lower than that observed with TEOS-derived gels. After one month in water, the radial shrinkage of a 14 mm diameter cylinder of gel prepared with DGS/PEO is about 10% after one month. This is the same shrinkage for the pure DGS gel. If the gel is aged in open system without water, the shrinkage is about 14% for the DGS/PEO gel, 21% for the DGS gel and 43% for the TEOS gel. Accordingly, the present invention relates to a method of preparing a bimodal meso/macroporous silica monolith with improved shinkage characteristics.

The formation of silica by a sol-gel route involves a complex series of hydrolyses and condensations.⁴¹ When multidentate starting materials are used, such as silanes derived from glycerol, sorbitol, mannitol, dextrans or other sugar-derived materials, the number of equilibria involved in the reaction cascade from starting materials to silica increases significantly. During this process, low molecular weight materials begin to oligomerize and polymerize. In the absence of significant amounts of other dopants, the final silica monolith forms an optically clear material that contains water, alcohols and other added dopants. The entire process occurs in one phase.

The expedient of adding water soluble polymers and other additives, such as compounds of Formula I which can participate in the sol gel chemistry, to the original sol complicates the evolution of the silica. The situation is reminiscent of dispersion polymerization, where after oligomerization, the growing polymer nucleates particle growth.⁴² In this case, the growing silica polymer precipitates from the sol while gelation continues. The specific timing, degree of polymerization, ultimate morphology (including size of the primary particles and aggregates, thickness of the binding silica layers, uniformity of the particle size, pore sizes and porosity) is affected by the quantity, molecular weight and specific molecular characteristics of the additives as shown above.

There are distinctions between the work described here and previous literature reports. These include the nature of the silicon-based starting materials and the interactions of the additives with them. First, the nature of the alkoxy groups on the silane precursors of the present invention gives these compounds very different pH cure profiles than silanes derived from mono-hydroxysilanes; the residual alcohols of the precursors of the present invention act to plasticize the developing silica network. They also provide an environment which is not destabilizing to entrapped protein. Another distinction is the thermal dependence of the reaction. Gelation occurs at ambient temperature over a wide pH range, again facilitating the incorporation of proteins and other biomolecules in the method of the present invention. Finally, the shrinkage of these monoliths of the present invention is significantly reduced when compared to TEOS- or TMOS-derived materials, again providing a more stable environment for entrained biomolecules.

The use of different additives, of different MW and quantities in the sol-gel silica recipe allows the possibility of tuning surface area, total porosity, morphology and protein retention of the resulting structure, and the magnitude of shrinkage and strength over wide ranges prepared by the sol-gel method from sugar alcohol and related silanes. Another advantage with this combination of reagents over traditional routes is the mild thermal conditions that can be used for its manufacture. In particular, the synthetic route is compatible with the incorporation of proteins and other biomolecules.

(ii) Uses

The siliceous materials prepared using the methods of the invention are novel accordingly, the present invention further includes all uses of these materials, including, but not limited to, their use in chromatography, biosensors, immobilizing enzymes, affinity supports and the like. In many applications for these materials, a biological substance has been entrapped within its matrixes.

Accordingly, the present invention includes the use of a siliceous material comprising an active biological substance entrapped therein, as biosensors, immobilized enzymes or as affinity chromatography supports. Therefore, the present invention also includes a method for the quantitative or qualitative detection of a test substance that reacts with or whose reaction is catalyzed by an active biological substance, wherein said biological substance is encapsulated within a siliceous material, and wherein said siliceous material is prepared using a method of the invention. The quantitative/qualitative method comprises (a) preparing the siliceous material comprising said active biological substance entrapped within a porous, silica matrix prepared using a method of the invention; (b) bringing said biological-substance-containing siliceous material into contact with a gas or aqueous solution comprising the test substance; and (c) quantitatively or qualitatively detecting, observing or measuring the change in one or more characteristics in the biological substance entrapped within the siliceous material or, alternatively, quantitatively or qualitatively detecting, observing or measuring the change in one or more characteristics in the test substance. Such tests may be performed in various morphologies that will be readily understood by those skilled in the art. Without limitation, these can include microarrays, such as would be achieved using a pinspotter.⁴³

In particular, the invention includes a method, wherein the change in one or more characteristics of the entrapped biological substance is qualitatively or quantitatively measured by spectroscopy, utilizing one or more techniques selected from the group consisting of UV, IR, visible light, fluorescence, luminescence, absorption, emission, excitation and reflection.

Also included is a method of storing a biologically active biological substance in a silica matrix, wherein the biological substance is an active protein or active protein fragment, wherein the silica matrix prepared using a method of the invention.

The bimodal meso/macroporous silica monoliths prepared using the method of the invention are especially useful in chromatographic applications. For the preparation of a chromatographic column, the silica precursor (optionally in hydrolyzed form) and water-soluble polymer (and other additives) may be placed into a chromatographic column before phase transition and gelation occurs.

The present invention therefore relates to a method of preparing a monolithic silica chromatographic column comprising placing a solution comprising an organic polyol silane precursor and one or more additives selected from water-soluble polymers and a compound of Formula I, wherein R⁴ is group selected from polymer-(linker)ₙ- and

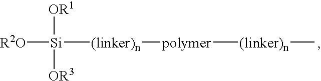

in a column under conditions suitable for a phase transition to occur before gelation.

Other additives known in the art for use with sol gel columns may also be used in the method of the invention. This includes, for example, substances, such as aminopropyltriethoxysilane (APTES), which provide cationic sites that counterbalance the anionic charge of the silica to reduce non-selective interactions. Other amino-functional materials described above PEG-NH₂, PPG-NH₂ and/or PAM, can similarly serve this role.

In embodiments of the invention the chromatographic column is a capillary column. Conventional capillary columns comprise a cylindrical article having an inner wall and an outer wall and involve a stationary phase permanently positioned within a circular cross-section tube having inner diameters ranging from 5 μm to 0.5 mm. The tube wall may be made of glass, metal, plastic and other materials. When the tube wall is made of glass, the wall of the capillary possesses terminal Si—OH groups which can undergo a condensation reaction with terminal Si—OH or Si—OR groups on the silica monolith to produce a covalent “Si—O—Si” linkage between the monolith and the capillary wall. This provides a column with structural integrity that maintains the monolith within the column. Due to the small dimensions of a capillary column, the solutions comprising the silica precursor and water soluble polymer may be introduced into the capillary by the application of a modest vacuum.

Some of the additives can be removed or eluted prior to chromatography by rinsing with an appropriate solvent, such as water and/or alcohol. The column may be further prepared by methods such as supercritical drying or the use of a reagent such as a silane or other coupling agent to modify the surface of the exposed silica. The monolith may also be stored with the additives interspersed within.

In embodiments of the invention, the silica monolith prepared using the method of the invention is further derivatized to allow tailoring of the monolith for a variety of chromatographic separations. For example, a surface may be incorporated into the monolith that is useful for reverse phase chromatography. Such surfaces may comprise long chain alkyl groups or other non-polar groups. Such derivatization may be done by reacting the Si—OH or Si—OR groups on the silica with reagents that convert these functionalities to surface linkages to other organic groups such as alkyls, aryls or functional organic groups (e.g. carboxylates or amines). In still further embodiments, the other organic groups are chiral molecules that facilitate the separation of chiral compounds. These derivatizations are known in the art and are included within the scope of the present invention.

The present invention also includes chromatographic columns comprising the silica monoliths prepared as described herein. Accordingly the invention includes a chromatographic column comprising a silica monolith prepared by combining an organic polyol silane precursor and one or more additives selected from water-soluble polymers and a compound of Formula I, wherein R⁴ is group selected from polymer-(linker)ₙ- and,

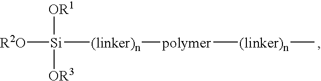

under conditions where a phase transition occurs before gelation.

In addition, the invention includes the use of a silica monolith prepared using a method of the invention and comprising an active biological substance entrapped therein, as chromatographic columns, biosensors, immobilized enzymes or as affinity chromatography supports. Therefore, the present invention relates to the use of a silica monolith comprising an active biological substance entrapped therein to quantitatively or qualitatively detect a test substance that reacts with or whose reaction is catalyzed by said encapsulated active biological substance, and wherein said silica monolith is prepared using a method of the invention.

Also included is a method for the quantitative or qualitative detection of a test substance that reacts with or whose reaction is catalyzed by an active biological substance, wherein said biological substance is encapsulated within a silica monolith, and wherein said silica monolith is prepared using a method of the invention. The quantitative/qualitative method comprises (a) preparing a silica monolith comprising said active biological substance entrapped within a porous, silica matrix prepared using the method of the invention; (b) bringing said biological-substance-comprising silica monolith into contact with a gas or aqueous solution comprising the test substance; and (c) quantitatively or qualitatively detecting, observing or measuring the change in one or more characteristics in the biological substance entrapped within the silica monolith or, alternatively, quantitatively or qualitatively detecting, observing or measuring the change in one or more characteristics in the test substance.

In particular, the invention includes a method, wherein the change in one or more characteristics of the entrapped biological substance is qualitatively or quantitatively measured by spectroscopy, utilizing one or more techniques selected from the group consisting of UV, IR, visible light, fluorescence, luminescence, absorption, emission, excitation and reflection.

(iii) Specific Application to Bioaffinity Chromatography

The present inventors have developed biocompatible, bimodal meso/macroporous silica materials that can be used for biomolecule (e.g. protein) entrapment and have shown that capillary columns based on this material can be prepared that are suitable for pressure driven liquid chromatography and are compatible with mass spectral (MS) detection. The columns were prepared using a mixture of the biomolecule-compatible silica precursor diglycerylsilane (DGS),^{6,44,45} polyethylene oxide (PEO, MW 10,000), which controls morphology, aminopropyltriethoxysilane (APTES), which provides cationic sites that counterbalance the anionic charge of the silica to reduce non-selective interactions,³⁴ and a buffered solution of the biomolecule of interest to provide bioaffinity sites within the column. The resulting sol mixture was loaded into fused silica capillaries (150-250 μm i.d.), whereupon phase separation of PEO occurred followed by gelation of the silica. The phase separation of the polymer from the silica resulted in a bimodal pore distribution which produced large macropores (>0.1 μm) to allow good flow of eluent with minimal backpressure, and mesopores (ca. 3-5 nm diameter) that retained a significant fraction of the entrapped protein.

Accordingly, the present invention relates to a method of preparing a monolithic silica column having an active biomolecule entrapped therein comprising combining:

a) a polyol-silane derived silica precursor;

b) one or more additives selected from water soluble polymers and a compound of Formula I, wherein R⁴ is group selected from polymer-(linker)ₙ- and;

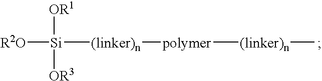

and

c) a biomolecule;

under conditions wherein a phase separation occurs before gelation.

In embodiments of the present invention, the additive is one or more water soluble polymers or compound of Formula I, wherein R⁴ is

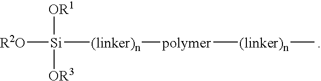

In further embodiments, R¹, R² and R³ are the same or different and are selected from C₁₋₄alkyl, in particular methyl or ethyl. In still further embodiments, linker is a C₁₋₄alkylene group, in particular a C₂₋₃alkylene group and n is 1. In still further embodiments, the water soluble polymer and the “polymer” group in R⁴ are both selected from PEO, polyNIPAM and PEO-NH₂.

In embodiments of the invention, the organic polyol silane silica precursor, one or more additives and biomolecule are also combined with a substance which provides cationic sites that counterbalance the anionic charge of the silica to reduce non-selective interactions, for example, aminopropyltriethoxysilane (APTES), PEG-NH₂, PPG-NH₂ and/or PAM. In further embodiments of the invention, the amount of a substance which provides cationic sites that counterbalance the anionic charge of the silica to reduce non-selective interactions is kept below levels which cause retention of anionic species. For example, when this substance is APTES, an amount in the range of about 0.2-0.4% (w/v), preferably about 0.3%, was found to be optimal for minimizing non-selective retention.

In embodiments of the present invention, the monolithic silica is prepared directly in a chromatographic column. The organic polyol silane silica precursor may be hydrolyzed, for example by dissolution in aqueous solution with optional sonication, and optionally in the presence of acid, for example 1M HCl, filtered to remove unwanted particulates if necessary, and the hydrolyzed precursor may then be combined with buffered solutions of the one or more additives, biomolecule and any further additives. In particular the hydrolyzed precursor may be combined with buffered solutions of one or more additives, biomolecule and a substance which provides cationic sites that counterbalance the anionic charge of the silica to reduce non-selective interactions, for example, aminopropyltriethoxysilane (APTES), PEG-NH₂, PPG-NH₂ and/or PAM. The resulting mixture may then be transferred to a column before phase separation and gelation occur. Preferably, the inner surface of the column is pre-treated with a substance to promote adhesion of the monolithic silica, for example aminopropyltriethoxysilane (APTES), PEG-NH₂, PPG-NH₂ and/or PAM. In embodiments of the invention, the column is a capillary column.

As a specific application of the new bioaffinity columns, the ability of small enzyme inhibitors to interact with an entrapped enzyme, and thus be retained on the column, was examined. The enzyme chosen for this study was the clinically relevant protein dihydrofolate reductase (DHFR). DHFR catalyzes the NADPH-dependent reduction of dihydrofolate (DHF) to tetrahydrofolate, which is then used as a co-factor in the biosynthesis of thymidylate, purines and several amino acids.^{46,47,48} DHFR is an essential enzyme in the cell and is the target for antifolate drugs.⁴⁹ A key reason for choosing this protein was that there are a large number of known DHFR inhibitors that span 5 decades of affinity, providing a useful model system for examining the binding of inhibitors to the entrapped enzyme.⁴⁹ This enzyme has also been shown to remain active and can bind to inhibitors when entrapped in DGS derived materials.⁴⁴

Examination of ligand binding was done via frontal affinity chromatography with mass spectrometric detection (FAC/MS). This method has recently been promoted as a potential high-throughput screening tool that is amenable to compound mixtures.¹⁷ The basic premise is that continuous infusion of a compound will allow for equilibration of the ligand between the free and bound states, where the precise concentration of free ligand is known. In this case, the breakthrough time of the compound will correspond to the affinity of the ligand for the immobilized biomolecule—ligands with higher affinity will break through later. As shown hereinbelow, DHFR loaded columns derived by the sol-gel method are suitable for FAC/MS based screening of ligand mixtures, and can be used to identify nanomolar inhibitors of the immobilized protein.

Formation of columns within fused silica capillaries, for example 150-250 μtm i.d. capillaries) provides a system that requires only very small amounts of protein (50 pmol loading, 12 pmol active protein) to produce a useful bioaffinity column. Such columns are suitable for pressure-driven liquid chromatography and can be operated at relatively high flow rates (up to 500 μL.min⁻¹) with low backpressures. More importantly, the operation of these columns with low ionic strength eluents allows direct interfacing to an electrospray mass spectrometer, allowing direct identification of small molecule identities using multiple reaction monitoring mode. The ability to detect inhibitors present in compound mixtures via retention time combined with MS detection may prove to be very powerful for high-throughput screening of compound mixtures. The extension of FAC/MS technology to entrapped proteins may improve the versatility of the FAC method, particularly since a wide range of proteins, including membrane-bound receptors,⁵⁰ can be entrapped in sol-gel derived silica.

The present invention further relates to a chromatographic column prepared by combining a polyol-silane derived silica precursor with one or more additives, a biomolecule and, optionally, a substance which provides cationic sites that counterbalance the anionic charge of the silica to reduce non-selective interactions, under conditions wherein a phase separation occurs before gelation. Also included within the scope of the present invention is the use of this column, for example but not limited to, in methods for immunoaffinity chromatography, sample cleanup, solid phase extraction or preconcentration of analytes, removal of unwanted contaminants (for example by antibody binding), solid phase catalysis and frontal affinity chromatography (with or without mass spectral detection).

The following non-limiting examples are illustrative of the present invention:

### EXAMPLES

### Materials and Methods for Examples 1-6

D-Gluconolactone (glulactone), D-maltose monohydrate, iodine, silver carbonate, 3-aminopropyltriethoxysilane (Aldrich Chemical Co.), anhydrous methyl sulfoxide and dextran (Sigma Chemical Co.) were used as received. The strong cationic exchange resin Amberlite IR-120 (Aldrich Chemical Co.) was rinsed with distilled water before use. D-Maltonolactone (maltolactone), dextran lactone (from dextran, average MW 10200) and dextran lactone (from dextran, average MW 43000) were prepared according to the literature.⁵¹ Poly(ethylene glycol) (average MW 200, 600, 2000, 10000) was purchased from Aldrich Chemical Co. Triethoxysilane, and allyl bromide were provided by Aldrich. Platinum-divinyltetramethyldisiloxane complex in vinyl-terminated polydimethylsiloxane, provided by Gelest Inc., was used as the Pt catalyst. Dichloromethane and pentane were distilled from CaH, EtOH was distilled from Mg before use.

¹H and ¹³C NMR were recorded at room temperature on a Bruker AC-200 spectrometer; solid state ¹³C and ²⁹Si CPMAS NMR spectra were recorded on a Bruker AC-300 at 75.47 and 59.62 MHz, respectively. FT-IR spectra were obtained on a Perkin-Elmer 283 spectrometer, samples were prepared as KBr pellets. Electrospray mass spectra were recorded on a Micromass Quattro LC, triple quadruple MS. Mean mobility data were recorded on a PALS Zeta Potential Analyzer Ver. 3.19. Thermogravimetric analyses were obtained using a Thermowaage Sta STA 409.

### Example 1

### Preparation of Silsesquioxane Precursors

GluconamideSi, 1. To a solution of D-gluconolactone (0.91 g, 5.2 mmol) in DMSO (10 mL) and EtOH (5 mL) was added 3-aminopropyltriethoxysilane (1.11 g, 5.0 mmol). The mixture was stirred at 60 C for 20 h. The solvents were evaporated under vacuum and oil residue was dissolved in dichloromethane. Unreacted D-gluconolactone was filtered off, the filtrate was concentrated and added to a large amount of pentane. The white precipitate was collected and dried in vacuo to give 1 as pale yellow solid, 1.83 g (92% yield). ¹H NMR (200.2 MHz, d₆-DMSO): δ 0.50 (SiCH₂), 1.12 (t, 9H, J=6.98 Hz, SiOCH₂CH₃), 1.45 (m, br, 2H, SiCH₂CH₂), 3.04 (m, 2H, CH₂NHCO), 3.74 (q, J=6.98 Hz, 6H, SiOCH₂CH₃), 3.40-5.32 (m, glucose ring CH and CH₂, and OH), 7.61 (s, br., 1H, NHCO). ¹³C NMR (50.3 MHz, d₆-DMSO): δ 7.8 (SiCH₂), 18.7-18.9 (SiOCH₂CH₃), 23.3 (SiCH₂CH₂), 41.5 (CH₂NHCO), 58.3 (SiOCH₂CH₃, overlapped), 64.0, 70.7, 72.1, 74.2, 73.0 (glucose ring CH and CH₂), 172.9 (NHCO). FT-IR (KBr): 1646 cm⁻¹ (v(C═O)). MS-ESI (ES⁺): 422.2 (M+Na, 100)⁺, 400.2 (M+1, 15)⁺, 354 (5), 236 (18).

MaltonamideSi, 2. To a solution of D-maltonolactone (0.75 g, 2.2 mmol) in DMSO (10 mL) and EtOH (5 mL) was added 3-aminopropyltriethoxysilane (0.44 g, 2.0 mmol). The mixture was stirred at 60 C for 20 h. The solvents were evaporated under vacuum and oil residue was dissolved in dichloromethane. Unreacted D-maltonolactone was filtered off, the filtrate was concentrated and added to a large amount of pentane. White precipitate was collected and dried in vacuo to give 2 as pale yellow solid, 0.98 g (87% yield). ¹H NMR (200.2 MHz, d₆-DMSO): δ 0.49 (m, br., 2H, SiCH₂), 1.08 (t, J=6.96 Hz, 9H, SiOCH₂CH₃), 1.43 (m, br., 2H, SiCH₂CH₂), 3.70 (q, J=6.96 Hz, 6H, SiOCH₂CH₃), 3.05-5.47 (m, CH₂NHCO and maltose CH and CH₂, and OH), 7.60 (NHCO) ppm. ¹³C NMR (50.3 MHz, d₆-DMSO): δ 7.8 (SiCH₂), 18.4˜19.1 (SiOCH₂CH₃), 23.4 (SiCH₂CH₂), 41.3 (CH₂NHCO), 56.6 (SiOCH₂CH₂), overlapped), 61.3, 63.4, 69.8, 72.5˜73.8 (overlapped), 80.6, 101.4 (maltose CH and CH₂), 172.9 (NHCO) ppm. FT-IR (KBr): 1643 cm⁻¹ (v(C═O)). MS-ESI (ES⁺): 584.3 (M+Na, 30)⁺, 562.4 (M+1, 20)⁺.

DextronamideSi-10K, 3a. To a solution of dextran10K-lactone (2.0 g, 0.2 mmol) in DMSO (50 mL) and EtOH (10 mL) was added 3-aminopropyltriethoxysilane (0.44 g, 2.0 mmol). The mixture was stirred at 60 C for 48 h. The mixture was concentrated and added to large amount of dichloromethane. White precipitate was collected, washed with dichloromethane, and dried in vacuo to give 3a as white solid, 1.8 g.

DextronamideSi-40K, 3b. To a solution of dextran40K-lactone (4.3 g, 0.1 mmol) in DMSO (50 mL) and EtOH (10 mL) was added 3-aminopropyltriethoxylsilane (0.44 g, 2.0 mmol). The mixture was stirred at 60 C for 48 h. The mixture was concentrated and added to large amount of dichloromethane. White precipitate was collected, washed with dichloromethane, and dried in vacuo to give 3b as white solid, 4.0 g.

### Example 2

### Preparation of PEO-Silyl Additives

[(CH₂CH₂O)ₚ(CH₂CH═CH₂)₂, 4a: The reaction was carried out under N₂ atmosphere. To a solution of poly(ethylene glycol) (average MW 200, 10.0 g, 50 mmol) in THF (100 mL) at 0 C was added NaH (2.4 g, 100.0 mmol) slowly over 30 min. The mixture was allowed to warm up to room temperature and stirred for 2 h. The mixture was cooled down to 0 C, allyl bromide (12.1 g, 100.0 mmol) was added. The mixture was warmed up to room temperature and stirred for further 15 h. White precipitate was filtered off and washed with THF (3×10 mL). Combined filtrate and washing solution and THF was evaporated to give pale yellow crude product. The crude product was purified by chromatography (SiO₂, 2% MeOH in CH₂Cl₂ as eluent) give allyl terminated poly(ethylene glycol), 4a as colorless oil, 11.1 g, (ca. 76% yield). ¹H NMR (200.2 MHz, CDCl₃): δ 3.54-3.62 (m, 18H, PEO OCH₂), 3.96 (dd, 1H, J=5.6 Hz, J=1.4 Hz, CH₂═CHCH₂O), 3.97 (dd, 1H, J=5.6 Hz, J=1.4 Hz, CH₂═CHCH₂O), 5.14 (m, 4H, CH₂═CHCH₂O), 5.86 (m, 2H, CH₂═CHCH₂O) ppm. ¹³C NMR (50.3 MHz, CDCl₃): δ 69.2, 70.4 (PEO OCH₂), 72.0 (CH₂═CHCH₂O), 116.9 (CH₂═CHCH₂O), 134.6 (CH₂═CHCH₂O) ppm. MS (EI), m/z, 275 (28, M, n=4), 319 (100, M, n=5), 363 (80, M, n=6), 407 (13, M, n=7), 451(5, M, n=8).

(CH₂CH₂O)ₚ(CH₂CH═CH₂)₂, ATPEO600: 4b: To a solution of poly(ethylene glycol) (average MW 600, 6.0 g, ca.10 mmol) in THF (100 mL) at 0 C was added NaH (0.50 g, 20.8 mmol) slowly over 15 min. The mixture was allowed to warm up to room temperature and stirred for 5 h. The mixture was cooled down to 0 C, allyl bromide (2.42 g, 20.0 mmol) was added. The mixture was warmed up to 40 C and stirred for further 3 h. White precipitate was filtered off and washed with THF (3×10 mL). Combined filtrate and washing solution, THF was evaporated to give pale yellow crude product. The crude product was purified by chromatography (SiO₂, 10% ethyl acetate in hexane as eluent) give allyl terminated poly(ethylene glycol), 4b as colorless oil, 6.1 g, (ca. 90% yield). ¹H NMR (200.2 MHz, CDCl₃): δ 3.54-3.65 (m, 44H, PEO OCH₂), 3.97 (dd, 2H, J=5.6 Hz, J=1.1 Hz CH₂═CHCH₂O), 3.98 (dd, 2H, J=5.6 Hz, J=1.1 Hz CH₂═CHCH₂O), 5.19 (m, 4H, CH₂═CHCH₂O), 5.88 (m, 2H, CH₂═CHCH₂O) ppm. ¹³C NMR (50.3 MHz, CDCl₃): δ 69.4, 70.5 (PEO OCH₂), 72.2 (CH₂═CHCH₂O), 117.1 (CH₂═CHCH₂O), 134.7 (CH₂═CHCH₂O) ppm. MS(maldi), m/z, 693 (5, M+Na⁺, n=13), 671(4, M+1, n=13), 649 (8, M+Na⁺, n=12), 627 (6, M+1, n=12), 605(12, M+Na⁺, n=11), 583(6, M+1, n=11), 561(9, M+Na⁺, n=10), 539 (5, M+1, n=10), 517(7, M+Na⁺, n=9), 495(5, M+1, n=9), 473 (6, M+Na⁺, n=8), 42.1(100).

(CH₂CH₂O)ₚ(CH₂CH═CH₂)₂, 4c: To a solution of poly(ethylene glycol) (average MW 2000, 2.0 g, ca.1 mmol) in THF (20 mL) at room temperature was added NaH (0.050 g, 2.1 mmol). The mixture was stirred at 50 C for 2 h. Allyl bromide (0.24 g, 2.0 mmol) was added. The mixture was stirred at room temperature for further 10 h. White precipitate was filtered off and washed with THF (3×10 mL). Combined filtrate and washing solution, THF was evaporated to give pale brown crude product. The crude product was purified by chromatography (SiO₂, CH₂Cl₂ as eluent) give allyl terminated poly(ethylene glycol), 4c as white solid, 1.89 g, (ca. 91% yield). ¹H NMR (200.2 MHz, CDCl₃): δ 3.50-3.65 (m, 180H, PEO OCH₂), 3.97 (dd, 2H, J=5.6 Hz, J=1.3 Hz CH₂═CHCH₂O), 3.98 (dd, 2H, J=5.6 Hz, J=1.3 Hz CH₂═CHCH₂O), 5.16 (m, 4H, CH₂═CHCH₂O), 5.83 (m, 2H, CH₂═CHCH₂O) ppm. ¹³C NMR (50.3 MHz, CDCl₃): δ 69.2, 70.3 (PEO OCH₂), 72.2 (CH₂═CHCH₂O), 116.9 (CH₂═CHCH₂O), 134.5(CH₂═CHCH₂O) ppm.

(CH₂CH₂O)ₚ(CH₂CH═CH₂)₂, 4d: To a solution of poly(ethylene glycol) (average MW 10K, 10 g, ca.1 mmol) in THF (100 mL) at room temperature was added NaH (0.050 g, 2.1 mmol). The mixture was stirred at 50 C for 2 h. Allyl bromide (0.24 g, 2.0 mmol) was added. The mixture was stirred at room temperature for further 10 h. White precipitate was filtered off and washed with THF (3×20 mL). Combined filtrate and washing solution, THF was evaporated to give pale brown crude product. The crude product was dissolved in dichloromethane (20 mL), added to large amount of diethyl ether to give white precipitate. Repeated precipitate procedure once more gave allyl terminated poly(ethylene glycol), 4d as white solid, 7.9 g, (ca. 77% yield). ¹H NMR (200.2 MHz, CDCl₃): δ 3.48-3.70 (m, 900H, PEO OCH₂), 3.96 (m, 4H, CH₂═CHCH₂O), 5.18 (m, 4H, CH₂═CHCH₂O), 5.84 (m, 2H, CH₂═CHCH₂O) ppm. ¹³C NMR (50.3 MHz, CDCl₃): δ 69.2-70.4 (PEO OCH₂), 72.1 (CH₂═CHCH₂O), 116.9 (CH₂═CHCH₂O), 134.6 (CH₂═CHCH₂O) ppm.

(CH₂CH₂O)ₚ[(EtO)₃Si(C₃H₆)]₂, 5a: To a mixture of 4a (1.98 g, 7.1 mmol) and triethoxylsilane (2.33 g, 14.2 mmol) one drop of Karstedt's Pt catalyst was added. The mixture was stirred at room temperature for 2h (the reaction was monitored by ¹H NMR). The volatile organics was removed at 100 C under vacuum. The residue was diluted with CH₂Cl₂ (50 mL), activated charcoal (0.5 g) was added, the mixture was stirred at room temperature overnight. After filtering through charcoal, CH₂Cl₂ was evaporated off to give 5a as colorless oil, 4.20 g, ca. 98% yield. FTIR(neat), v (cm⁻¹) 2975s, 2929s, 2885s, 1635w, 1443m, 1391s, 1366w, 1296w, 1262w, 1195m, 1167s, 1106s, 1082s, 959s, 793s, 694w; ¹H NMR (200.2 MHz, CDCl₃): δ 0.57 (m, 4H, SiCH₂), 1.17 (t, 18H, J=7.0 Hz, SiOCH₂CH₃), 1.60(m, 4H, SiCH₂CH₂CH₂), 3.38(m, 4H, SiCH₂CH₂CH₂), 3.52-3.60 (m, 18H, PEO OCH₂), 3.78 (dd, 4H, J=7.0 Hz, J=14.0 Hz, SiOCH₂CH₃) ppm. ¹³C NMR (50.3 MHz, CDCl₃): δ 6.5(SiCH₂), 18.4(SiOCH₂CH₃), 23.0(SiCH₂CH₂CH₂), 58.4(SiOCH₂CH₃), 70.1, 70.7 (PEO OCH₂), 73.7 (SiCH₂CH₂CH₂) ppm.

(CH₂CH₂O),[(EtO)₃Si(C₃H₆)]₂, 5b: To a mixture of 4b (2.1 g, ca. 3 mmol) and trietboxylsilane (1.2 g, 6.9 mmol) one drop of Karstedt's Pt catalyst was added. The mixture was stirred at room temperature for 1 h (the reaction was monitored by ¹H NMR). The volatile organics was removed at 110 C under vacuum. The residue was diluted with CH₂Cl₂ (50 mL), activated charcoal (0.5 g) was added, the mixture was stirred at room temperature overnight. After filtering through charcoal, CH₂Cl₂ was evaporated off to give 5b as colorless oil, 2.45 g, ca. 80% yield. FTIR(neat), v (cm⁻¹) 2975s, 2928s, 2884s, 2741w, 1741w, 1631w, 1459m, 1445m, 1391m, 1352w, 1297w, 1257w, 1107s, 1083s, 959m, 794m, 699w; ¹H NMR (200.2 MHz, CDCl₃): δ 0.61 (m, 4H, SiCH₂), 1.20 (t, 18H, J=7.1 Hz, SiOCH₂CH₃), 1.64(m, 4H, SiCH₂CH₂CH₂), 3.41(m, 4H, SiCH₂CH₂CH₂), 3.57-3.63 (m, 56H, PEO OCH₂), 3.80 (dd, 4H, J=7.1 Hz, J=14.0 Hz, SiOCH₂CH₃) ppm. ¹³C NMR (50.3 MHz, CDCl₃): δ 6.5(SiCH₂), 18.4(SiOCH₂CH₃), 23.0(SiCH₂CH₂CH₂), 58.5(SiOCH₂CH₃), 70.1, 70.7 (PEO OCH₂), 73.8 (SiCH₂CH₂CH₂) ppm.

(CH₂CH₂O)ₚ[(EtO)₃Si(C₃H₆)]₂, 5c: To a mixture of 4c (2.0 g, ca. 1 mmol) and triethoxylsilane (0.36 g, 2.2 mmol) in dichloromethane (20 mL) one drop of Karstedt's Pt catalyst was added. The mixture was stirred under refluxing for 3 h (the reaction was monitored by ¹H NMR). The solvent was evaporated and thereafter the volatile organics was removed at 110 C under vacuum. The residue was diluted with CH₂Cl₂ (50 mL), activated charcoal (0.5 g) was added, the mixture was stirred at room temperature overnight. After filtering through charcoal, CH₂Cl₂ solution was concentrated and thereafter added to large amount of diethyl ether to give 5c as colorless solid, 2.1 g, ca. 88% yield. FTIR(neat, KBr), v (cm⁻¹)

2975s, 2929s, 2885s, 1633w, 1459m, 1391s, 1366w, 1296w, 1262w, 1257w, 1194m, 1167s, 1106s, 1082s, 959s, 794s, 698w ¹H NMR (200.2 MHz, CDCl₃): δ 0.89 (m, 4H, SiCH₂), 1.18 (t, 18H, J=7.1 Hz, SiOCH₂CH₃), 1.54(m, 4H, SiCH₂CH₂CH₂), 2.65(m, 4H, SiCH₂CH₂CH₂), 3.49-3.72 (m, 188H, PEO OCH₂ and SiOCH₂CH₃, overlapped) ppm. ¹³C NMR (50.3 MHz, CDCl₃): δ 10.4(SiCH₂), 18.1(SiOCH₂CH₃), 22.6(SiCH₂CH₂CH₂), 58.1(SiOCH₂CH₃), 69.2-70.2, overlapped (PEO OCH₂ and SiCH₂CH₂CH₂) ppm.

(CH₂CH₂O)ₚ[(EtO)₃Si(C₃H₆)]₂, 5d: To a mixture of 4d (5 g, ca. 0.5 mmol) and triethoxylsilane (0.18 g, 1.1 mmol) in dichloromethane (50 mL) one drop of Karstedt's Pt catalyst was added. The mixture was stirred refluxing for 5 h (the reaction was monitored by ¹H NMR). The solvent was evaporated and thereafter the volatile organics was removed at 110 C under vacuum. The residue was diluted with CH₂Cl₂ (100 mL), activated charcoal (1.0 g) was added, the mixture was stirred at room temperature overnight. After filtering through charcoal, CH₂Cl₂ solution was concentrated and thereafter added to large amount of diethyl ether to precipitate white solid. Repeated precipitation procedure gave 5d as white solid, 2.7 g, ca. 50% yield. FTIR(neat, KBr), v (cm⁻¹) 2974s, 2929s, 2885s, 1631w, 1454m, 1391s, 1364w, 1266w, 1257w, 1167s, 1082s, 959m, 794m, 698w; ¹H NMR (200.2 MHz, CDCl₃): δ 0.66 (m, 4H, SiCH₂), 1.20 (m, 18H, SiOCH₂CH₃), 1.56(m, 4H, SiCH₂CH₂CH₂), 2.65(m, 4H, SiCH₂CH₂CH₂), 3.20-3.90 (m, 910H, PEO OCH₂ and SiOCH₂CH₃, overlapped) ppm. ¹³C NMR (50.3 MHz, CDCl₃): δ 6.2(SiCH₂), 18.0(SiOCH₂CH₃), 23.0(SiCH₂CH₂CH₂), 58.2(SiOCH₂CH₃), 68.8-70.3, overlapped (PEO OCH₂ and SiCH₂CH₂CH₂) ppm.

### Example 3

### Preparation of DGS/modified PEO gel

DGS (0.2648 g, 1.27 mmol) was mixed with (EtO)₃Si(CH₂)₃PEO(CH₂)₃Si(OEt)₃ (Example 3, 0.1274 g, 0.053 mmol) and added with water (600 μL, 33.3 mmol). The mixture was sonicated at 0 C for 1.5 h during which time a turbid solution formed. Then TRIS buffer (600 μL, 50 mM, pH=8.4) was added. The gel formed starting at the bottom of the solution after 5 min.

### Example 4

### Preparation of Samples 6-15

All of the following samples were treated in the following way after gelation: Fresh sol-gels were aged in a closed container at 5 C for 20 h, then further aged at room temperature for 7 or 20 days. Aged hydrogels were washed with water 5×5 mL. This was done by soaking the whole aged gel (1 mL initial volume) in 5 mL water at room temperature for 4 h. The water was replaced 4 times, the last time the gel was kept over 8 h, for a total of 24 h. The gels were then allowed to dry at room temperature in a opened container for 45 days. Shrinkage was recorded against the initial volumes of the sample sols. The results are shown in FIG. 5.

(a) Sample 6. To a solution of DGS (240 mg, 1.1 mmol) in H₂O (0.50 mL) was added Tris buffer (0.50 mL, 50 mM, pH=8.4). The mixture left at room temperature to gel (Table 1). The hydrogel was then aged at 5 C for 20 h in a closed container, then further aged and dried in air at room temperature for 6 days. The gel was washed with water, and then allowed to dry at room temperature in an open container for 45 days. Shrinkage was then recorded. Freeze drying gave a colorless solid.

(b) Sample 7. To a solution of DGS (240 mg, 1.1 mmol) in H₂O (0.50 mL) was added sorbitol (60 mg, 0.33 mmol in 0.50 mL (50 mM, pH=8.4) Tris Buffer). The mixture was left at room temperature to gel (Table 1). The hydrogel was aged at 4 C for 20 h in a closed container, then further aged and dried in air at room temperature for 6 days. The gel was washed with water and then allowed to dry at room temperature in an open container for 45 days. Shrinkage was then recorded. Freeze drying gave a white powder.

(c) Samples 8-15 : Prepared in a similar manner. The reaction conditions are listed in Table 1.

### Example 5

### Mobility Measurement: Zeta Potential

After freeze drying, samples 6-11 were ground into powder. Colloidal dispersions were made by adding silica powder to Tris buffer solution (as shown in Table 3), which were transferred to a cuvette for mobility measurement.

Instrument parameters: wavelength=661.0 nm; field frequency=5.00 Hz; voltage=10.00 volts; electric field=25.45 V/cm. Results are shown in Table 3.

### Example 6

### Shrinkage and Swelling

The change in volume from the original sol volume of the samples over 45 days was measured on a volume/volume % basis. The results are shown in FIG. 5.

### Materials and Methods for Examples 7-18

DGS was synthesized using methods previously reported.⁶ The poly(ethylene oxide) (PEO) used was provided by Aldrich and had an average MW of ca. 10,000 and 100,000, respectively. Poly(N-isopropyl acrylamide) (pNIPAM) was provided by Polysciences, Inc. and had a molecular weight of 17,000 and 65,000 poly(ethylene glycol) terminated by amino groups (PEG-NH₂) was provided by Nektar Therapeutics with molecular weight of 3400. Poly(propylene glycol)bis(2-amino-propyl ether) was provided by Aldrich and had a molecular weight of 230 and 400, respectively. The molecular weight determined by GPC was 104,000 (Mn, with polystyrene as calibrant). Human serum albumin (HSA) was obtained from Sigma and was fluorescently labeled with FITC as previously described.⁵² Human serum album (HSA), lysozyme and a Lowry protein assay kit (P5656) were also provided by Sigma.

DSC

The differential scanning calorimeter (DSC) analysis was carried out on a TA 2100 Modulated Differential Scanning Calorimeter at a heating rate of 15 C /min under nitrogen atmosphere.

TGA

Thermogravimetric analysis was performed using a THERMOWAAGE STA409. The analysis was measured under air, with flow rate of 50 cc/min. The heat rate was 5° C./min starting at room temperature.

Porosity BET

The surface area, pore volume and pore radius were measured with an Autosorb 1 machine from Quantachrome. The samples were evacuated to 100 millitorr before heating. The vacuum was maintained during the outgassing at 200 C with a final vacuum on the order of 10 millitorr (or less) at completion of the outgassing. The samples were backfilled with nitrogen for removal from the outgas station and prior to analysis. BET surface area was calculated by BET (Brunauer, Emmett and Teller) equation; the pore size distribution and pore radius nitrogen adsorption-desorption isotherms was calculated by BJH (Barrett, Joyner and Halenda) method. All the data were calculated by the software provided with the instruments.

Electron Microscopes

The sample was observed by JEOL 840 Scanning Electron Microscopy (SEM) and JEOL Transmission Electron Microscope.

Confocal Microscopy Images to Examine HSA within the Gels

Gels entrapped with FITC-labeled HSA solution were made in vials and Petri dishes. After washing, very thin films of the gels were used for confocal microscopy to examine the areas of labeled HSA within the gels. The images were taken with a Zeiss LSM 510 Confocal Microscope.

UV-Visible Spectrophotometer

A gel was prepared with DGS/PEO/FITC-labeled HSA as described above. The gel was washed with 0.05 M NaHCO₃ and the washings were examined by a Cary 400 Bio UV-visible Spectrophotometer after centrifugation to get rid of the (gel) particulate.

### Example 7

### Preparation of DGS Gel

DGS (0.50 g, 2.40 mmol) was dissolved into water (500 μL, 27.8 mmol) with sonication at 0 C until it completely dissolved. TRIS buffer (500 μL, 10-50 mM, pH=8.35) was added. The time when the solution lost its ability to flow was recorded as gel time (t_{gel}).

### Example 8

### Preparation of DGS/PEO Gel

PEO (MW=100,000) was dissolved into TRIS buffer (1.0 mL, 10-50 mM, pH=8.35); solutions of different concentrations were prepared. DGS (0.50 g, 2.40 mmol) was dissolved into water (500 μL, 27.8 mmol), and sonicated at 0 C until it totally dissolved. The PEO solution (500 AL) was added. Macroporous gels arose when PEO solutions of concentration 0.01-0.08 g/mL were used to make the sol. The time required for the solution to become totally opaque was recorded as phase separation time (tₚₛ), and the time when the opaque phase lost its ability to flow was recorded as gel time (t_{gel}) (Table 1). After gelation, the gel was soaked in water (5 mL) for 12 h and then stored in fresh water or allowed to dry in air at room temperature. BET data is provided in Table 10.

FIG. 7 provides a graph showing the gel time of DGS doped with different concentrations of PEO prepared in an analogous manner as described in this example.

### Example 9

### Preparation of DGS/PEO/PPG-NH₂ Gel

0.5 g PEO (MW=10,000) was dissolved into phosphate buffer (1.0 mL, 5-10 mM, pH=7.5-8.5); 0.5 g PPG-NH₂ (MW=230) was dissolved into phosphate buffer (1.0 mL, 5 mM, pH=7.5); solutions of different ratio of PEO/PPO-NH₂ were prepared (see Table 9). DGS (0.50 g, 2.40 mmol) was dissolved into water (500 μL, 27.8 mmol), and sonicated at 0 C until it totally dissolved. 200 ILL DGS solution was added with 60 μL PEO/PPG-NH₂ solution. The time required for the solution to become totally opaque was recorded as phase separation time (tₚₛ), and the time when the opaque phase lost its ability to flow was recorded as gel time (t_{gel}) (Tables 1 and 10). After gelation, the gel was soaked in water (5 mL) for 12 h and then stored in fresh water or allowed to dry in air at room temperature

### Example 10

### Preparation of DGS/polyNIPAM Gel

polyNIPAM was dissolved into water (50 mg NIPAM/1000μL H₂O). DGS (0.50 g, 2.40 mmol) was dissolved into water (500 μL, 27.8 mmol) with sonication at 0 C until it was totally dissolved. The polyNIPAM solution (500 μL) was then added, and the solution mixed thoroughly to give a sol containing 0.025 g/mL of polyNIPAM. After gelation, the gel was soaked in water (10 mL) for 12 h and then stored in fresh water or allowed to dry in air at room temperature.

### Example 11

### Preparation of DGS/PEO—NH₂ and DGS/PEO/PPG-NH₂

PEO—NH₂ (MW=3,400) was dissolved into Phosphate buffer (1.0 mL, 5-50 mM, pH 7-8.5); solutions of different concentrations were prepared. DGS (0.50 g, 2.40 mmol) was dissolved into water (500 μL, 27.8 mmol), and sonicated at 0° C until it totally dissolved. The PEO—NH₂ solution (500 μL) was added. Macroporous gels arose when PEO—NH₂ solutions of concentration larger than 0.05 g/mL were used to make the sol. The time required for the solution to become totally opaque was recorded as phase separation time (tₚₛ), and the time when the opaque phase lost its ability to flow was recorded as gel time (t_{gel}). After gelation, the gel was soaked in water (5 mL) for 12 h and then stored in fresh water or allowed to dry in air at room temperature.

A similar process was used to prepare gels doped with both PEO and PPG-NH₂. Several stock aqueous solutions of PEO and PPG-NH₂ were prepared (Table 9). DGS (1.001 g) was dissolved in DGS dissolved in distilled water (1 mL) with sonication over about 20 minutes. To the DGS solution (200 μl) was added the “Polymer Mixture” with stirring. The sol was then allowed to gel (Table 10).

### Example 12

### Gels with Entrapped Protein

These gels were prepared as described in Examples 8, 10-12 except that the protein (HSA) was dissolved into the polymer/buffer solution prior to addition to the DGS solution (10 mg HSA/1000 μL solution, i.e. 0.5 g DGS, 5 mg HSA, 25 mg PEO, 1000 μL water).

### Example 13

### Calculating the Amount of PEO Left in Gels After Washing Using Thermogravimetric Analysis

DGS and DGS/PEO monoliths were formed by pouring off the excess liquid after phase separation and gelation had occurred. The gels were washed 3 times by soaking in water, each time with 20 mL water, for 1 day. The gels could be washed as a monolith, or after crushing to give comparable results. The washed gels were dried in open air for 2 days, then freeze-dried for more than one day. The sample was first exposed to vacuum in a flask cooled with dry ice and then at RT. Graphs indicate there is roughly 24% PEO left in the gels after washing (FIG. 10).

### Example 14

### Finding the Structure of PEO when in Gels by Usage of a Differential Scanning Calorimeter

DSC was used to measure the thermal properties and structures of the DGS and DGS/PEO gel (FIG. 11).

### Example 15

### Confocal Microscopy Images to Examine HSA within the Gels

Gels prepared from DGS (0.5 g), water (0.5 mL), and PEO (0.5 mL of a 0.05 g PEO/1 mL buffer (10 mM Tris buffer) solution) and FITC-labelled HSA solution consisting of 0.750 mL PEO and 0.250 mL labeled HSA) were made in vials and in Petri dishes. After washing, the location of labeled HSA within the gels was determined, in very thin films of the gels prepared using a razor blade, by confocal microscopy (FIG. 14).

### Example 16

### Preparation of Gels for BET Analysis

Two gels prepared from TEOS (0.5 g), aqueous HCl (pH 1.6, 0.5 mL of 0.024 M solution) and Tris buffer (0.5 mL, pH=8.25) were made for BET analysis with gel times of 6.5 and 6 minutes, respectively.

Gels prepared from DGS (0.5 g), water (0.5 mL) and PEO (MW 100,000, 0.5 mL of a 0.05 g/mL solution) were also made for BET analysis with phase separation times of 3 minutes and gelation times of 7 minutes, respectively.

### Example 17

### Testing Loss of HSA from Gels with UV-Visible Spectrophotometer

A gel was prepared with DGS/PEO/FITC-labeled HSA as described above (i.e. 0.5 g DGS, 500 μL H₂O, 750 μL PEO solution [50 mg PEO Mw 100000/1000 μL 10 mM Tris buffer solution], 1000 μL FITC labeled HSA solution [10 mg HSA/1000 μL 10 mM Tris buffer solution]). It phase separated at 1.5 min and gelled at 3 min. The gel was washed 3 times with 20 mL 0.05 M NaHCO₃, each time for 24 h, and the washings were tested using UV spectroscopy. The intensity of the peaks due to the FITC label on the HSA peaks became much smaller with each subsequent washing. However, the washings were contaminated with particles, which reduced the sensitivity of the method. Therefore, the Lowry Method of protein detection was also used.⁵³ Fluorescein isothiocyanate (FITC, Aldrich) was used to label the proteins. Labeling was carried out in pH 9.5 carbonate buffer (0.05 M) for 2 h at 5° C. Dilutions of HSA with PEO and HSA with Tris buffer were made to form an HSA standard curve using fluorescein). The first set of gels contained 0.5 g DGS in 0.5 mL water, and 0.5 mL of an HSA/PEO solution of 10 mg HSA and 1 mL varying concentrations of PEO (MW 100,000). The gels were washed (with water) on the day of gel preparation (1^{st} washing), the day after gel preparation (2^{nd} washing), and the 4^{th} day after gel preparation (3^{rd} washing). The protein content of the washings was determined using the Lowry method as described below.

Determination of Protein Concentration by Lowry Method:

5.0-10.0 mg of protein was entrapped within gels prepared with 0.5 g DGS. After gelation, 20 ml 5-10 mM phosphate buffer were added three times, soaking the gel. The buffer is changed every 24 hours. All the washings and gels were kept at 4° C. in a refrigerator. The washings were measured by Lowry method with the reagents proved from Sigma (Sigma Protein Assay Kit, procedure No. P5656). The standard curves were plotted using HSA, BSA and lysozyme as standards respectively. The measurements were performed in 96-well plates using a TECAN Safire absorbance/fluorescence plate reader operated in absorbance mode at 750 nm.

These data are reported in Tables 7 and 8 HSA and lysozyme respectively, where it is evident that more protein was washed out when the PEO concentration is high and that PPG-NH₂ is much more efficient and retaining proteins than other polymers.

### Materials and Methods for Examples 18-20

Chemicals

Tetraethylorthosilicate (TEOS, 99.999%) was obtained from Aldrich (Oakville, ON). Diglyceryl silane precursors were prepared from TEOS as described below. Human serum albumin (HSA), trimethoprim, pyrimethamine, folic acid, polyethyleneglycol (PEG/PEO, MW 2K to 100K) and fluorescein were obtained from Sigma (Oakville, ON). Coumarin was obtained from Molecular Probes Inc. (Eugene, Oreg.). Dihydrofolate reductase (from E. coli) was provided by Professor Eric Brown (McMaster University). Fused silica capillary tubing (150-250 μm i.d., 360 μm outer diameter, polyimide coated) was obtained from Polymicro Technologies (Phoenix, Ariz.). All water was distilled and deionized using a Milli-Q synthesis A10 water purification system. All other reagents were of analytical grade and were used as received.

Preparation of DGS

TEOS was distilled to remove any residual water and a neat mixture of the anhydrous TEOS (2.08 g, 10.0 mmol) and glycerol (1.84 g, 20.0 mmol) was heated at 130 C for 36 h, during which time EtOH was distilled off. Complete removal of EtOH and unreacted starting materials at 140 C in vacuo gave DGS as a solid compound that was not contaminated with residual ethanol. Structural characterization of DGS by NMR and the properties of DGS derived silica are reported elsewhere.⁴⁵

Preparation of Columns

Prior to loading of columns the inner surface of the fused silica capillary was coated with APTES to promote adhesion of the monolithic silica column. The capillary was first washed with 3-4 volumes of: 1 M NaOH; H₂O; 1 M HCl; H₂O and EtOH. At this point, 1 mL of 2% (v/v) APTES in absolute EtOH was loaded into the column and left to react for 12 hr at 110 C, after which the excess APTES was washed out with water and the capillary was dried for 12 hr at 110 C.

Silica sols were prepared by first mixing 1 g of DGS (finely ground solid) with 990 μL of H₂O and optionally 10 μL of 1 M HCl to yield ˜1.5 mL of hydrolyzed DGS, after 15-25 min of sonication. The hydrolyzed DGS was filtered through a 0.45 μm syringe to remove particulates before use. A second aqueous solution of 50 mM HEPES at pH 7.5 was prepared containing 16% (w/v) PEO (MW=10 000) and 0.6% (v/v) APTES. This aqueous solution also contained ca. 20 μM of DHFR. 100 μL of the Buffer/PEG/APTES/DHFR solution was mixed with 100 μL of hydrolyzed DGS and the mixture was immediately loaded via syringe pump into a fused silica capillary (ca. 2 m long, 150-250 μm i.d.). The final composition of the solution was 8% w/v PEO (10K), 0.3% v/v APTES and 10 μM DHFR in 25 mM HEPES buffer. The mixture underwent spinodal decomposition (phase separation) in about 2-3 min followed by silica polymerization (˜10 min) to generate a hydrated macroporous monolithic column containing entrapped protein. After loading the sol-gel mixture, the monolithic columns were aged for a minimum of 5 days at 4 C and then cut into 10 cm lengths before use. In some cases, the APTES present in the columns was replaced with a final concentration of either 0.03% polyallylamine (PAM, MW 17,000) or 0.3% dimethyldimethoxysilane (DMDMS) to examine the effects of surface derivatization on non-selective retention.

Characterization of Silica Morphology

The morphology of the column was assessed using nitrogen adsorption porosimetry (for characterization of mesopores) or scanning electron microscopy (SEM) for characterization of macropores. Pore-size analysis of completely dried monoliths was performed on a Quantachrome Nova 2200 surface area/pore-size analyzer. Before analysis, the monoliths were washed copiously to remove any entrapped glycerol, were crushed to a fine powder, freeze-dried and outgassed at 120 C for 4 hours to remove air and bound water from the surface of the powder. The pressure was measured as nitrogen was adsorbed and desorbed at a constant temperature of −196° C. Using the desorption branch of the resulting isotherm the average pore-size and distribution of pore-sizes was determined using the BJH (Barrett, Joyner and Halenda) calculation.⁵⁴ SEM analysis was done by cutting the capillary to expose a fresh surface, which was then coated with a gold film under vacuum to improve conductivity. Imaging was performed at 10 kV using a JEOL 840 Scanning Electron Microscope.

FAC/MS Studies

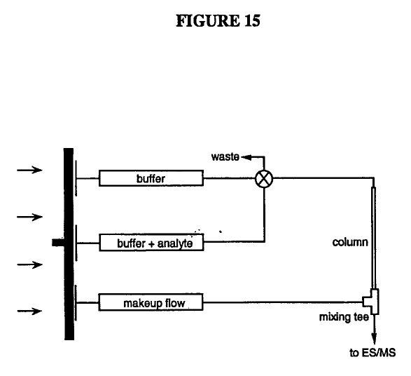

The frontal affinity chromatography system/mass spectrometer system is shown in FIG. 15. Syringe pumps (Harvard Instruments PhD 2000) were used to deliver solutions, and a flow-switching valve was used to toggle between the assay buffer and the solution containing the compound mixture. This solution was then pumped through the column to achieve equilibrium. Effluent was combined with suitable organic modifiers to assist in the generation of a stable electrospray and detectability of the sprayed components using a triple-quadrupole MS system (Sciex API 3000). This configuration allows for maximum flexibility in compound introduction. Full operation of FAC-MS methods requires frequent flow switching between two solutions connected to the head of the column. An Upchurch microinjection valve allows syringe contents to be exchanged during operation. Columns were interfaced to the FAC system using Luer-capillary adapters (Luer Adapter, Ferrule and Green Microtight Sleeve from Upchurch (P-659, M-100, F-185X)). All other connections between components were achieved using fused silica tubing.

Typical FAC/MS experiments involved infusion of mixtures of compounds containing 1-200 nM of each compound, including coumarin and fluorescein as void markers, folic acid (micromolar inhibitor) and pyrimethamine and trimethoprim (nM inhibitors). Before the first run, the column was flushed with 0.05 M NH₄OAc buffer (pH 6.6, 100 mM NaCl) for 30 min at different flow rates (from 1 to 5 μL.min⁻¹) to remove any glycerol and non-entrapped protein and then equilibrated with 2 mM NH₄OAc for 30 min at different flow rates (from 1 to 5 μL.min⁻¹). All compounds tested were present in 2 mM NH₄OAc and were delivered at a rate of 5 μL.min⁻¹ using the syringe pump. The makeup flow (used to assist in the generation of a stable electrospray) consisted of methanol containing 10% (v/v) NH₄OAc buffer (2 mM) and was delivered at 5 μL.min⁻¹. The mass spectrometer was operated in multiple reaction monitoring (MRM) mode with simultaneous detection of m/z 147_m/z 103 (coumarin), CE 25; m/z 249_m/z 177 (pyrimethamine), CE 40; m/z 291_m/z 230 (trimethoprim), CE 35; m/z 333_m/z 287 (fluorescein), CE 45 and m/z 442_m/z 295 (folic acid), CE 15.

Characterization of Column Performance

Columns of 10 cm length were prepared containing no protein (blanks), 50 pmol active DHFR, 50 pmol of DHFR that was partially denatured by boiling prior to use or 50 pmol of HSA (selectivity control). In all cases FAC/MS measurements were performed using the five compound mixture described above and the resulting frontal chromatograms were used to evaluate non-selective interactions of compounds with the column, the reversibility of binding, the potential for regeneration of columns and the level of leaching of entrapped protein.

Columns that contained active DHFR were further characterized by monitoring the breakthrough volume (obtained by multiplying flowrate by breakthrough time) as a function of analyte concentration using either pyrimethamine or trimethoprim. In each case, the data were fit to the following equation:¹⁷

$$V = {V}_{0} + \frac{{B}_{t}}{[ A ] + {K}_{d}} \qquad ( 1 )$$

where V₀ is the void volume (μL), V is the retention volume (μL), [A] is the analyte concentration (μM), K_{d} is the binding constant of the ligand to the protein (μM) and Bₜ is the total picomoles of active protein in the column.

### Example 18

### Column Formation and Optimization

It was critical that the bioaffinity columns be fabricated using protein-compatible processes, thus several issues were addressed to produce a viable monolithic bioaffinity column. Key goals to achieve when developing monolithic bioaffinity columns were: 1) to produce a biocompatible column matrix that entrapped biomolecules in an active form; 2) to have spinodal composition occur after column loading but before gelation of the silica phase to promote macroporosity; 3) to avoid shrinkage and cracking of the column, which would introduce unwanted flow channels; 4) to minimize protein leaching after gelation of the silica, and; 5) to minimize non-selective interactions between small molecules and the silica matrix. A variety of parameters were optimized to achieve this goal, including the silica precursor (TEOS vs. DGS), silica concentration (1-10 mol %), gelation pH (5 to 8), ionic strength (0 to 100 mM), and PEO concentration (2-12% w/v) and molecular weight (2 kDa-100 kDa). While several compositions produced viable columns, the best performance was obtained using a composition derived from the protein compatible precursor DGS which contained an initial level of 3.3 mol % Sio₂. Lower levels led to columns that would slowly dissolve in the mobile phase, while higher levels gelled too quickly to allow facile column loading. Optimal gelation conditions were achieved under mild conditions at 4° C., pH˜7 with an ionic strength of 25 mM. Optimal macroporosity was obtained using 8% w/v of 10 kDa PEO. Phase separation occurred for molecular weight values of 10 kDa or higher, and at levels of 2% w/v or higher for 10 kDa or higher molecular weight PEO. An optimal level of 8% w/v for 10K PEO was selected owing to the good homogeneity and reproducibility obtained for forming columns using this composition, and because higher levels or molecular weights of PEO produced solutions that were too viscous to allow facile loading of the column.

Early versions of columns used untreated, NaOH, methacryloxypropyltrimethoxysilane or 3-glycidoxypropyltrimethoxysilane-treated capillaries as supports. However, it was often observed that the monolith could be pushed out of the capillary at higher flow rates. To overcome this problem the inner surface of the capillary was pretreated with APTES, which provided a good bond between the monolith and the capillary surface. In such columns, flow rates as high as 500 μL.min⁻¹ could be achieved with no occurrences of monolith detachment from the capillary.

### Example 19

### Column Characterization

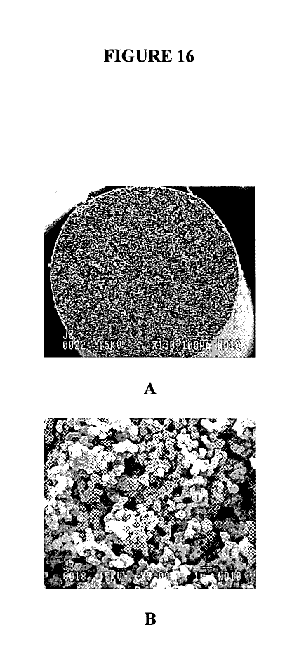

FIG. 16 shows scanning electron microscopy images of the DGS/PEO/APTES monolithic silica stationary phase. Panel A shows an image of a 1.0 mm diameter column that had been extruded from an uncoated capillary, and shows that the silica forms a self-supporting monolith. Panel B shows a high magnification image of a monolith within a 250 μm capillary, showing the macroporous nature of the silica skeleton. The silica matrix appears to be composed primarily of silica beads that are 1-2 μm in diameter and are linked together to form a continuous monolith. The voids (through-pore spaces) are on the order of a few microns in diameter, and provide sufficient void volume to allow good flow of liquids with low backpressure. Overall, the macroporous morphology of the columns appears to be quite similar to that reported by Tanaka for reversed phase columns (skeleton size of 1-2 μm, through-pore diameter of 2-8 μm), although it is important to note that in the case of Tanaka's columns the PEO was removed by pyrolysis before imaging.

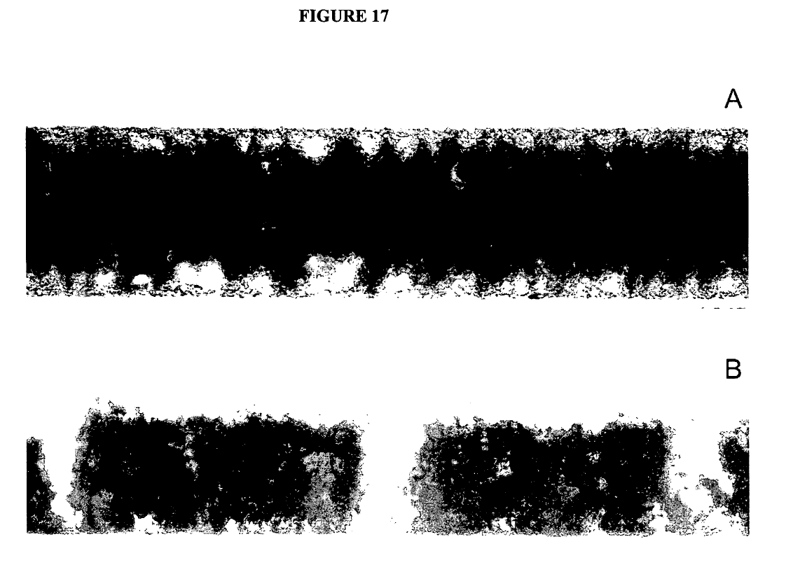

Attempts to image monoliths within 150-250 μm i.d. capillary columns via SEM showed that the introduction of the columns to ultrahigh vacuum (UHV) produced pullaway of the monolith from the capillary wall. To avoid UHV, the monoliths were imaged using brightfield microscopy. FIG. 17 shows a brightfield image of a filled capillary (250 μm i.d.) after 3 months of aging in buffer (Panel A), and clearly shows that the monolith completely fills the capillary with no pullaway. Panel B shows the same monolith after 24 hrs. of storage in a dessicator. Upon removal of entrapped water, the silica monolith shrinks significantly and exhibits cracking and pullaway. These results show that columns must be stored in a wet state to maintain column integrity. Such storage conditions are also necessary to maintain the activity of entrapped proteins.

BET measurements were performed on PEO doped samples to assess the morphology of the mesopores within the silica skeleton (note: measurements were done only for samples that were not pyrolyzed). Table 11 shows the mean pore diameter, surface area and volume occupied by mesopores within the column. It is evident that the addition of 10 kDa PEO leads to only minor decreases in surface area relative to pure DGS, but that the presence of PEO dramatically alters the fraction of mesopores (2-50 nm diameter) relative to micropores (<2 nm) in favor of mesopores. The addition of PEO also produces a higher total pore volume and a slightly larger average pore diameter, both of which should result in somewhat better flow properties. When considered together with the SEM data, it is apparent that the columns have the desired meso/macroporous morphology, although at this point we have not yet optimized the through-pore size and skeleton size of the monolithic silica columns.

### Example 20

### Bioaffinity Column Performance

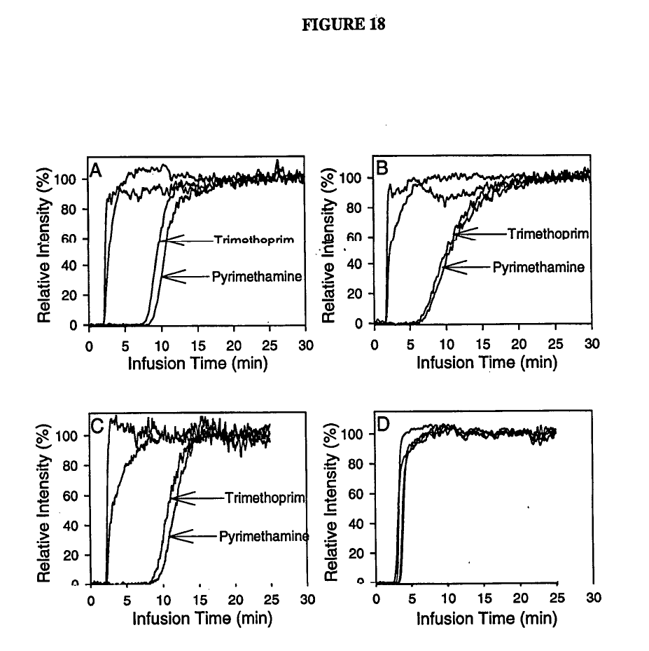

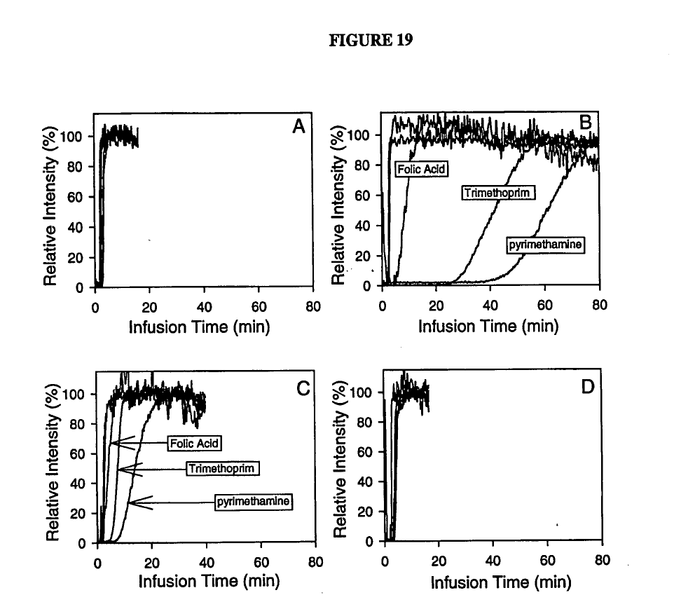

A key consideration in the development of bioaffinity columns for FAC/MS applications is to minimize non-selective adsorption of analytes to the column matrix while maximizing the retention of compounds owing to selective binding to the entrapped protein. FIG. 18 shows frontal chromatograms of unmodified columns relative to columns containing dimethyldimethoxysilane, aminopropylsilane or poly(allylamine)-derivatized silica. These additives allowed us to examine charged and uncharged additives and to modify the hydrophobicity of the column so as to modulate interactions of analytes with the silica. As shown in Panel A, the unmodified silica has a tendency to retain cationic species (pyrimethamine and trimethoprim) but does not retain either anionic or neutral species. Addition of either DMDMS or PAM did not significantly alter the retention properties, possibly owing to the low levels at which these could be employed before reducing column performance. However, even low levels of APTES led to almost complete removal of interactions between the silica matrix and cationic analytes, while retaining the low degree of non-selective adsorption of anionic and neutral species, in agreement with previous observations by Zusman for sol-gel based glass fiber affinity columns.³⁴ Higher levels of APTES caused retention of anionic species, and thus 0.3% APTES was found to be optimal for minimizing non-selective retention. Presumably, this level of APTES provides a net neutral surface that is somewhat amphiphlic owing to the propyl chains associated with APTES. Importantly, this surface maintained its ability to block non-selective retention over a period of months, indicating that the APTES formed a stable surface coating that did not change in composition with time. FIG. 19 shows FAC/MS traces obtained for elution of mixtures of DHFR inhibitors and control compounds through DGS/PEO/APTES columns containing no protein, active DHFR, partially denatured DHFR or HSA, a protein that does not bind DHFR inhibitors. The blank column shows the expected breakthrough of all compounds in the first few minutes, indicative of minimal non-selective interactions. Panel B shows significant retention of the two DHFR inhibitors, trimethoprim (K_{d}=4 nM, elution time of 39 min) and pyrimethamine (K_{d}=45 nM, retention time 55 min), less retention of a weak inhibitor (folic acid, K_{d}=11 μM, retention time=7 min) and no retention of non-selective ligands (fluorescein, coumarin, retention time=2 min). This result indicates that DHFR is active when entrapped in the column, in agreement with recent results from our group showing good activity of DHFR when entrapped in DGS derived materials.⁴⁴ Upon boiling DHFR prior to entrapment, all DHRF-binding ligands show significantly reduced retention times, consistent with partial denaturation of the protein. It should be noted that DHFR is known to be remarkably stable and that the thermal unfolding of DHFR is partially reversible,⁵⁵ and thus it is not surprising that partial activity is retained even after heat denaturation. As a secondary control, a column containing entrapped HSA was examined, and as shown in Panel D there is essentially no binding beyond that obtained in a blank column, indicating that the retention of the ligands is consistent with selective interactions between the ligands and DHFR. The reversal in the expected elution times for trimethoprim and pyrimethamine (based on their respective K_{d} values) is not fully understood at this time, but may be related to differences in on and off rates, which are likely to play a significant role in determining the overall retention time of compounds on the column. This is being examined in further detail and will be discussed in a future manuscript.

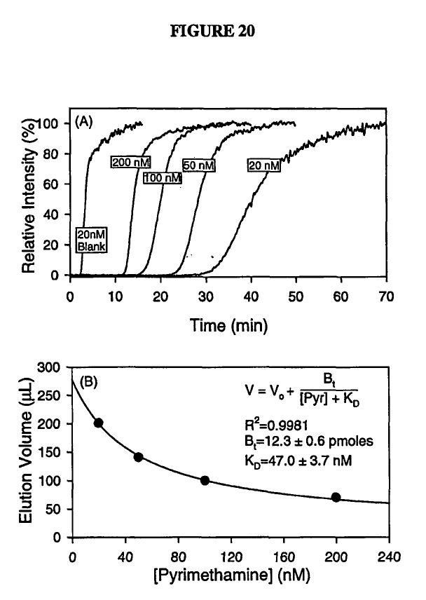

To further explore the properties of the DHFR-doped columns, the effect of ligand concentration on retention time was examined for both pyrimethamine and trimethoprim. As the concentration of ligand increases, one expects the column to saturate more rapidly for a given flow rate, and thus the compound is expected to breakthrough earlier. By plotting elution volume against analyte concentration one can determine the amount of protein immobilized (Bₜ) and the dissociation constant of the protein directly on the column. FIG. 20 shows breakthrough curves for pyrimethamine at various concentrations, and the resulting plot of V vs. [A]. From this data one extracts a total protein concentration of 12 pmol on the column, and a K_{d} of 47 nM. The K_{d} value is essentially identical to that in solution (37 nM) and is in excellent agreement with the value obtained for DHFR entrapped in DGS derived materials (46 nM).⁴⁴ The data obtained from trimethoprim provided a K_{d} value of 21 nM and a Bₜ value of 7 pmol (data not shown). The K_{d} value for trimethoprim in DGS is 3 nM, thus, the affinity of the inhibitor is somewhat lower than previously reported, but is still in the nM range and therefore would be considered a “hit” in a high-throughput screen. The Bₜ values indicate that approximately 75-85% of the initial protein present was either denatured, inaccessible or removed from the column during column conditioning. The latter possibility is not surprising given the significant fraction of macropores, and suggests that the fraction of protein that is retained may be present either in mesopores and/or tightly adsorbed to the surface of macropores. Given that the APTES coated surface does not appear to interact strongly with either cationic or anionic compounds, we speculate that the protein is likely to be entrapped in mesopores that are accessible to the analytes, although at this point we cannot exclude the possibility that some fraction of protein may be associated with the surface of macropores.

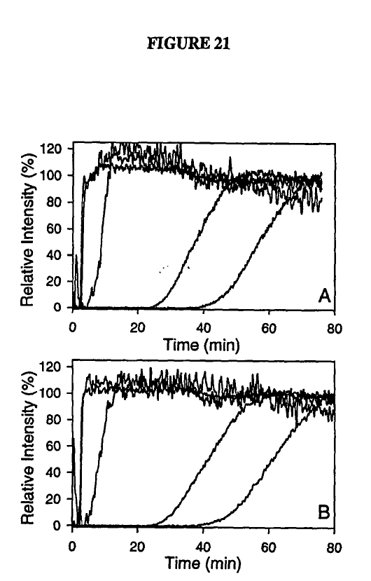

FIG. 21 shows the reproducibility between columns within the same batch (i.e., cut from the same capillary). In this case, 10 cm columns were cut from the midsection of 1 m long capillaries and were examined after washing 10 bed volumes of buffer through the column to remove glycerol and any loosely adsorbed protein. The data are all obtained for the first run of compounds through the columns. It is clear that the columns show excellent reproducibility, with the relative standard deviation between columns being in the range of 5% or less. Reproducibility between columns obtained from different batches was slightly poorer, showing RSD values on the order of 8% (data not shown). These data suggest that the sol-gel composition and processing methods used to form the column lead to reproducible column performance, and make it possible to directly compare data obtained from different columns. This is further supported by the data shown in FIG. 6, where data obtained from four different columns was combined to generate reliable K_{d} and Bₜ values with r²>0.998.

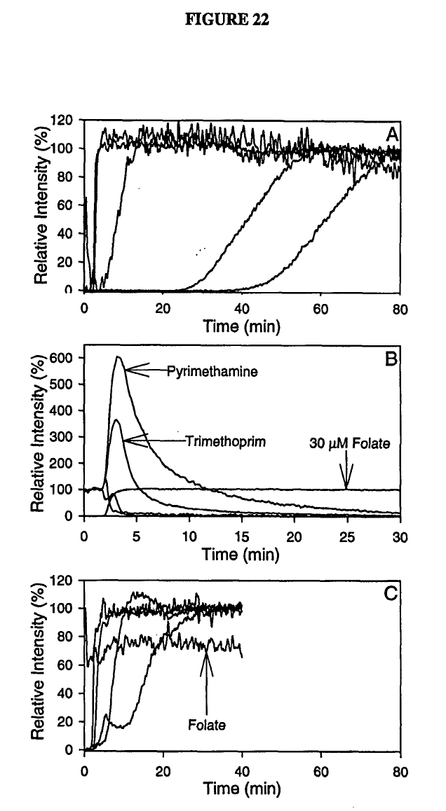

FIG. 22 shows the reproducibility between runs (Panels A and B) and the regenerability of the columns (Panel C) when using a weak affinity ligand (folic acid) to displace stronger binding ligands (pyrimethamine and trimethoprim). The retention time for pyrimethamine drops from 60 min to 15 min while those for trimethoprim drop from 40 min to 7 min after column regeneration. Thus the binding of a strong ligand to the column results in essentially irreversible loss of protein function, which cannot be recovered even after washing the column with an excess of a weak ligand to aid in displacement of stronger ligands. Similar behaviour has been reported for antibody-based columns, where binding of high-affinity antigens or haptens tends to lead to irreversible loss of column function.^{13f} While it may be possible to regenerate DHFR columns using alternative methods, such as chaeotropic reagents (3 M KSCN), denaturants (urea or guanidinium hydrochloride) or pH shifts, it is likely that any such regeneration methods will lead to irreversible denaturation of at least some fraction of the entrapped protein, which in turn will produce alterations in breakthrough times.

### Discussion for Examples 18-20

As demonstrated above, meso/macroporous sol-gel based monolithic bioaffinity columns are ideally suited for the screening of compound mixtures using frontal affinity chromatography with mass spectrometric detection for identification of specific compounds in the mixture. The ability to interface the capillary columns directly to an electrospray (ESI) mass spectrometer is a key advantage of the new columns, and is likely to make them suitable for HTS of compound mixtures using FAC/MS. While direct comparison to bead-based columns was not done in the present study, the monolithic columns clearly provide advantages in terms of ease of column loading and control over protein loading. Columns were formed simply by mixing the hydrolyzed silane with the polymer and protein-doped buffer and pumping the mixture into the capillary prior to spinodal decomposition and gelation. This one-step column fabrication method leads to good column-to-column reproducibility. The monolithic columns retained up to 25% of the loaded protein in an active form. The monolithic columns also have low backpressures (due to the macroporous nature of the material), which allows the use of a low-pressure syringe pump for pumping of eluents. The ability to operate at low pressures and low flowrates makes the monolithic columns amenable to direct interfacing with ESI/MS, with no need for flow splitting. This maximizes sensitivity and thus results in an ability to use low levels of compounds and hence small amounts of immobilized protein (ca. 10 pmol). This latter point is likely to be of significant importance when expensive and/or low abundance proteins are used as targets for FAC/MS based screening. Library compounds may be equally valuable and available in small quantities, making this technique more attractive.

One of the major advances in the development of the new columns was the use of the biocompatible sol-gel precursor DGS for column fabrication. Recent studies from our group have conclusively demonstrated that DGS and related sugar-modified silanes are able to maintain the activity of a wide variety of proteins, and in particular are able to stabilize proteins that denature readily when entrapped in materials derived form alkoxysilanes such as tetraethylorthosilicate.⁴⁴ The evolution of glycerol as a byproduct of DGS hydrolysis maintains the entrapped proteins in a active state during column aging, yet is readily removed from the column during the initial column flushing step, avoiding elution of glycerol into the mass spectrometer.

A key issue that was examined as part of column optimization was minimization of non-selective retention mechanisms which could result from interactions of compounds with the silica matrix. Since silica is polar and anionic, it is expected that interactions with polar and cationic compounds might occur, as was observed in our work. Counterbalancing of the anionic charge using the cationic silane APTES resulted in a remarkable reduction in non-selective retention, while at the same time not producing significant changes in entrapped protein behaviour. APTES could be easily incorporated into the column by adding it to a buffered PEO/protein solution, and the level could be adjusted simply by altering the APTES concentration in the starting buffer mixture.

An issue that remains to be addressed is regenerability, although as mentioned above, this problem is ubiquitous in immunoaffinity chromatography^{13a} after binding of high-affinity ligands, and may require significant efforts to identify conditions that can dissociate bound ligands without irreversibly denaturing the entrapped protein. In all likelihood, these conditions would have to be optimized for each new protein. Fortunately, it is possible to prepare several columns with good reproducibility using a single length of capillary and thus it is possible that rather than regenerating columns one could simply use new columns for each run and compare intercolumn runs to obtain Bₜ or K_{d} data, if needed.

While the current work has focused on entrapment of a soluble enzyme, the sol-gel method employed herein is also amenable to the entrapment of a wide range of important drug targets, including membrane-bound enzymes and receptors²¹ and even whole cells.⁵⁶ Furthermore, entrapment into DGS derived materials allows immobilization of labile enzymes, such as Factor Xa and Cox-II, which are difficult to immobilize by other methods.⁴⁴ Thus, the monolithic columns may find use in screening of compound mixtures against a wide variety of useful targets.

While the present invention has been described with reference to the above examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

Full Citations for Documents Referred to in the Specification

¹ A very clear discussion of the differences in flow characteristics between different types of silica may be found in Leinweber, F. C.; Lubda, D.; Cabrera, K.; Tallarek, U. Anal. Chem. 2002, 74, 2470.

² Nogues; J.-L.; Balaban; C.; Moreshead; W. V. U.S. Pat. No. 5,076,980 (to Geltech), 1991.

³ Premstaller, A.; Huber, C.; Oberarcher, H. World Patent Application WO0155713, Method and Apparatus for Separating Polynucleotides Using Monolithic Capillary Columns.

⁴ Control of pore size in macroporous sol gels: Tanaka, N.; Nagayama, H.; Kobayashi, H.; Ikegami, T.; Hosoya, K.; Ishizuka, N.; Minakuchi, H.; Nakanishi, K.; Cabrera, K.; Lubda, D. J. High Resol. Chromatogr. 2000, 23, 111.

⁵ (a) Merck column;

http://www.chromolith.com/english/services/chromatographie/hplc/chromolith/intro.html. (b) Nakanishi; K.; Soga; N.; Minakuchi; T. PCT publication number WO98/29350 (to Merck Patent GmbH), 1998.

⁶ Brook, M. A.; Brennan, J. D.; Chen, Y. Polyol-Modified Silanes as Precursors for Silica, U.S. Provisional Patent S.No. 60/384,084 (to McMaster University), Filed May 31, 2002 and PCT patent application S.No. PCT/CA03/00790, Filed Jun. 2, 2003.

⁷ Kikuta, K.; Ohta, K.; Takagi, K. Chem. Mater. 2002, 14, 3123.

⁸ Kang, J.; Wistuba, D.; Schurig, V. Electrophoresis 2002, 23, 1116.

⁹ Pope, E. J. A.; Sano, Y.; Wang, S.; Sarkar, A. U.S. Pat. No. 5,023,208 (to Orion Laboratories Inc.), 1991.

¹⁰ Ishizuka, N.; Minakuchi, H.; Nakanishi, K.; Hirao, K.; Tanaka, N. Coll. Surf. A: Physicochem. Eng. Asp. 2001, 187-188, 273. (b) Ishizuka, N., Minakuchi, H., Nakanishi, K., Soga, N., Nagayama, H., Hosoya, K., Tanaka, N. Anal. Chem. 2000, 72, 1275 and references cited therein.

¹¹ Nakanishi, K.; Soga, N.; Minakuchi, K. U.S. Pat. No. 6,531,060, Issued Mar. 11, 2003; Nakanishi, K.; Soga, N.; Minakuchi, K. U.S. Pat. No. 6,207,098, Issued Mar. 27, 2001; Nakanishi, K.; Soga, N.; U.S. Pat. No. 5,624,875, Issued Apr. 29, 1997.

¹² Brinker, C. J.; Scherer, G. W. Sol-Gel Science, Academic Press: San Diego, 1990.

¹³. a) Hage, D. S. J. Chromatog. B, 1998, 715, 3-38 b) Hage, D. S.; Clin. Chem. 1999, 45, 593-615; c) Weller, M. G. Fresenius J. Anal. Chem. 2000, 366, 635-645; d) Muronetz, V. I.; Sholukh, M.; Korpela, T. J. Biochem. Biophys. Methods 2001, 49, 29-47; e) Baczek, T.; Kaliszan, R. J. Biochem. Biophys. Methods 2001, 49, 83-98; f) Burgess, R. R.; Thompson, N. E. Curr. Opin. Biotechnol. 2002, 13, 304-308.

¹⁴. a) Hage, D. S.; Noctor, T. A. G.; Wainer, I. W. J. Chromatogr. A, 1995, 693, 23-32; b) Hofstetter, H.; Hofstetter, O. Schurig, V. J. Microcolumn September 1998, 10, 287-291; c) Hofstetter, O.; Lindstrom, H.; Hofstetter, H. Anal. Chem. 2002, 74, 2119-2125. Fitos, I.; Visy, J.; Simonyi, M. J. Biochem. Biophys. Methods 2002, 54, 71-84.

¹⁵. a) Hsieh, Y. L.; Wang, H.; Elicone, C.; Mark, J.; Martin, S. A.; Regnier, F. Anal. Chem., 1996, 68, 455-462; b) Wang, C.; Oleschuk, R.; Ouchen, F.; Li, J.; Thibault, P.; Harrison, D. J. Rapid. Commun. Mass. Spectrom. 2000, 14, 1377-1383; c) Wang, S.; Regnier, F. E. J. Chromatogr. A. 2001, 913, 429-436; d) Peterson, D. S.; Rohr, T.; Svec, F.; Frechet, J. M. J. J. Proteome Res. 2002, 1, 563-568; e) Peterson, D. S.; Rohr, T.; Svec, F.; Frechet, J. M. J. Anal. Chem. 2002, 74, 4081-4088; f) Slysz, G. W.; Schriemer, D. C. Rapid Commun. Mass Spec. 2003, 17, 1044-1050.

¹⁶. Prazeres, D.; Miguel F.; Cabral, J. M. S. in: Multiphase Bioreactor Design, Cabral, J. M. S.; Mota, M.; Tramper, J. (Eds.) Taylor & Francis Ltd., London, UK, 2001, pp. 135-180.

¹⁷. a) Schriemer, D. C.; Bundle, D. R.; Li, L.; Hindsgaul, O. Angew. Chem., Int. Ed. Engl. 1998, 37, 3383; b) Zhang, B.; Palcic, M. M.; Schriemer, D. C.; Alvarez-Manilla, G.; Pierce, M.; Hindsgaul, O. Anal. Biochem. 2001, 299, 173-182.

¹⁸. a) Baynham, M. T.; Patel, S.; Moaddel, R.; Wainer, I. W.; J. Chromatogr. B 2002, 772, 155-161; b) Moaddel, R.; Lu, L.; Baynham, M.; Wainer, I. W. J. Chromatogr. B 2002, 768, 41-53; c) Moaddel, R.; Cloix, J.-F.; Ertem, G.; Wainer, I. W. Pharmaceut. Res. 2002, 19, 104-107; d) Moaddel, R.; Wainer, I. W.; J. Pharmaceut. Biomed. Anal. 2003, 30, 1715-1724.

¹⁹. a) Royer, C. A. Prog. Biotechnol. 2002, 19, 17-25; b) Seemann, H.; Winter, R; Royer, C. A. J. Molec. Biol. 2001, 307, 1091-1102.

²⁰. Gill, I., Chem. Mater. 2001, 13, 3404.

²¹. Jin, W.; Brennan, J. D. Anal. Chim. Acta 2002, 461, 1.

²². Zheng, L.; Reid, W. R.; Brennan, J. D. Anal. Chem. 1997, 69, 3940.

²³. Zheng, L.; Brennan, J. D. Analyst 1998, 123, 1735.

²⁴. Edmiston, P. L.; Wambolt, C. L.; Smith, M. K.; Saavedra, S. S. J. Coll. Int Sci. 1994, 163, 395.

²⁵. Jordan, J. D.; Dunbar, R. A.; Bright, F. V. Anal. Chem. 1995, 67, 2436.

²⁶. Gottfried, D. S.; Kagan, A.; Hoffman, B. M.; Friedman, J. M. J. Phys. Chem. B 1999, 103, 2803.

²⁷. Doody, M. A.; Baker, G. A.; Pandey, S.; Bright, F. V. Chem. Mater. 2000, 12, 1142.

²⁸. Wambolt, C. L.; Saavedra, S. S. J. Sol-Gel Sci. Tech. 1996, 7, 53.

²⁹. Shen, C.; Kostic, N. M. J. Amer. Chem. Soc. 1997, 119, 1304.

³⁰. a) Braun, S.; Shtelzer, S.; Rappoport, S.; Avnir, D.; Ottolenghi, M. J. Non-Cryst. Solids, 1992, 147, 739; b) Avnir, D.; Braun, S.; Lev, O.; Ottolenghi, M. Chem. Mater. 1994, 6, 1605; c) Wang, R; Narang, U.; Prasad, P. N.; Bright, F. V. Anal. Chem. 1993, 65, 2671; d) Ellerby, L. M.; Nishida, C. R.; Nishida, F.; Yamanaka, S. A.; Dunn, B.; Valentine, J. S.; Zink, J. I. Science, 1992, 225, 1113; e) Wu, S.; Ellerby, L. M.; Cohan, J. S.; Dunn, B.; El-Sayed, M. A.; Valentine, J. S.; Zink, J. I. Chem. Mater. 1993, 5, 115; f) Dave, B. C.; Soyez, H.; Miller, J. M.; Dunn, B.; Valentine, J. S.; Zink, J. I. Chem. Mater. 1995, 7, 1431; g) Yamanaka, S. A.; Nishida, F.; Ellerby, L. M.; Nishida, C. R.; Dunn, B.; Valentine, J. S.; Zink, J. I. Chem. Mater. 1992, 4, 495; h) Dave, B. C.; Dunn, B.; Valentine, J. S.; Zink, J. I. Anal Chem. 1994, 66, 1120A; i) Blyth, D. J.; Aylott, J. W.; Richardson, D. J.; Russell, D. A. Analyst 1995, 120, 2725; j) Aylott, J. W.; Richardson, D. J.; Russell, D. A. Analyst 1997, 122, 77; k) Williams, A. K.; Hupp, J. T. J. Am. Chem. Soc. 1998, 120, 4366.

³¹. a) Braun, S.; Rappoport, S.; Zusman, R.; Avnir, D.; Ottolenghi, M. Mater. Lett. 1990, 10, 1; b) Heichal-Segal, O.; Rappoport, S.; Braun, S.; Biotechnology, 1995, 13, 798; c) Reetz, M. T.; Zonta, A.; Simpelkamp, J.; Biotechnol. Bioengin. 1996, 49, 527; d) Narang, U.; Prasad, P. N.; Bright, F. V.; Kumar, K.; Kumar, N. D.; Malhotra, B. D.; Kamalasanan, M. N.; Chandra, S. Chem. Mater. 1994, 6, 1596; e) Narang, U.; Prasad, P. N.; Bright, F. V.; Ramanathan, K.; Kumar, N. D.; Malhotra, B. D.; Kamalasanan M. N.; Chandra, S. Anal. Chem. 1994, 66, 3139; f) Jordan, J. D.; Dunbar, R. A.; Bright, F. V.; Anal. Chim. Acta 1996, 332, 83; g) Yamanaka, S. A.; Dunn, B.; Valentine, J. S.; Zink, J. I. J. Am. Chem. Soc. 1995, 117, 9095; h) Kauffinann, C.; Mandelbaum, R. T.; J. Biotechnol. 1998, 62, 169.

³². a) Bronshtein, A.; Aharonson, N.; Avnir, D.; Turniansky, A.; Altstein, M. Chem. Mater. 1997, 9, 2632; b) Altstein, M.; Bronshtein, A.; Glattstein, B.; Zeichner, A.; Tamiri, T.; Almong, J. Anal. Chem. 2001, 73, 2461; c) Bronshtein, A.; Aharonson, N.; Turniansky, A.; Altstein, M. Chem. Mater. 2000, 12, 2050; d) Cichna, M.; Knopp, D.; Niessner, R. Anal. Chim. Acta 1997, 339, 241; e) Cichna, M.; Markl, P.; Knopp, D.; Niessner, R. Chem. Mater. 1997, 9, 2640; f) Schedl, M.; Wilharm, G.; Achatz, S.; Kettrup, A.; Niessner, R.; Knopp, D. Anal. Chem. 2001, 73, 5669-5676; g) Spitzer, B.; Cichna, M.; Markl, P.; Sontag, G.; Knopp, D.; Niessner, R. J. Chromatogr. A 2000, 880, 113.

³³. Cichna, M. J. Sol-Gel Sci. Technol. 2003, 26, 1159-1164.

³⁴. Zusman R.; Zusman, I. J. Biochem. Biophys. Methods 2001, 49, 175-187.

³⁵. a) Sakai-Kato, K.; Kato, M.; Toyo'oka, T. Anal. Chem. 2002, 74, 2943-2949; b) Kato, M.; Sakai-Kato, K; Matsumoto, N.; Toyo'oka, T. Anal. Chem. 2002, 74, 1915-1921; c) Sakai-Kato, K.; Kato, M.; Toyo'oka, T. Anal. Biochem. 2002, 308, 278-284; d) Sakai-Kato, K.; Kato, M.; Nakakuki, H.; Toyo'oka, T. J. Pharmaceut. Biomed. Anal. 2003, 31, 299-309; e) Sakai-Kato, K; Kato, M.; Toyo'oka, T. Anal. Chem. 2003, 75, 388-393; f) Kato, M.; Matsumoto, N.; Sakai-Kato, K.; Toyo'oka, T. J. Pharmaceut. Biomed. Anal. 2003, 30, 1845-1850.

³⁶. Macbeath, G.; Schreiber, S. L. Science 2000, 289, 1760.

³⁷ Brook, M. A.; Brennan, J. D.; Chen Y. PCT patent application S. No. PCT/CA03/00790, filed Jun. 3, 2003.

³⁸ (a) Gluconamides: (i) DePasquale, R. J.; Wilson, M. E. U.S. Pat. No. 4,591,652 (to SCM Corp., USA), 1986. (ii) Haupt, M.; Knaus, S.; Rohr, T.; Gruber, H. J. Macromol. Sci., Part A: Pure Appl. Chem. 2000, A37, 323. (b) A related compound, the maltopentaose-modified silane: Enomoto, N.; Furukawa, S.; Ogasawara, Y.; Akano, H.; Kawamura, Y.; Yashima, E.; Okamoto, Y. Anal. Chem. 1996, 68, 2798.

³⁹ Brook, M. A. Silicon in Organic, Organometallic, and Polymer Chemistry, Wiley: N.Y., 2000, Chap. 1.

⁴⁰ Flora, K. K.; Brennan, J. D.; Baker, G. A.; Doody, M. A.; Bright, F. V. Biophys. J. 1998, 75, 1084.

⁴¹ (a) Brinker, C. J.; Scherer, G. W. Sol-Gel Science, Academic Press: San Diego, 1990. (b) Iler, R. K. The Chemistry of Silica, Wiley: N.Y., 1979. (c) Brook, M. A. Silicon in Organic, Organometallic, and Polymer Chemistry, Wiley: N.Y., 2000, Chap 10.

⁴² Stöver, H. D. H.; Li, K. Dispersion Polymerization, in “Polymeric Materials Encyclopedia”, Salamone, J. C., Ed. CRC Press: Boca Raton, Fla. USA, 1996, Vol. 3, pp. 1900.

⁴³ Brennan, J.; Rupcich, N. Multicomponent Protein Microarrays, U.S. Provisional Patent Application S.No. 60/422,892, filed Nov. 1, 2002.

⁴⁴. Besanger, T. R.; Chen, Y; Deisingh, A. K; Hodgson, R.; Jin, W.; Mayer, S.; Brook, M. A.; Brennan, J. D. Anal. Chem. 2003, 75, 2382-2391.

⁴⁵. Brook, M. A.; Chen, Y.; Guo, K.; Zhang, Z.; Brennan, J. D. Adv. Mater. 2003, submitted.

⁴⁶. Hitchings, G. H. Angew. Chem. Int. Ed. Engl. 1989, 29, 879.

⁴⁷. Stone, S. R.; Morrison, J. F. Biochemistry 1988, 27, 5493.

⁴⁸. Benkovic, S. J.; Fierke, C. A.; Naylor, A. M. Science 1988, 239, 1105.

⁴⁹. Polshakov, V. I.; Biekofsky, R. R.; Birdsall, B.; Feeney, J. J. Mol. Struct. 2002, 602-603, 257. ⁵⁰ John D. Brennan, Michael A. Brook and Travis Besanger, Method of Immobilizing Membrane-Associated Molecules, U.S. Provisional Patent Application S.No. 60/426,018 (to McMaster University) Filed Nov. 14, 2002.

⁵¹ (a) Hamshimoto, K.; Imanishi, S.; Okada, M.; Sumitomo, H. J. Polym. Sci., Polym. Chem. Ed. 1991, 29, 1271; (b) Zhang, T.; Marchant, R. E. Macromolecules 1994, 27, 7302.

⁵² Zelisko, P. M.; Brook, M. A. Langmuir 2002, 18, 8982.

⁵³ (a) Lin, Y.; Hlady, V. Colloids Surf., B 1994, 2, 481. (b) Hasegawa, M.; Gitano, H. Langmuir 1992, 8, 1582.

⁵⁴. Barrett, E. P.; Joyner, L. G. Halenda, P. H. J. Amer. Chem. Soc. 1951, 73, 373.

⁵⁵. Iwakura, M.; Honda, S. J. Biochem. 1996, 119, 414-420.

⁵⁶. Livage, J.; Coradin, T.; Roux, C. J. Phys.: Condens. Matter 2001, 13, R673.

TABLE 1
Preparation of TQ resins

| | Ratio of DGS: | | | |
|---|---|---|---|---|
| | Gluconamide-Si(OEt)₃ | Gelation time | Aged | Yield |
| Sample | (w/w) | (min)^{b} | (days) | (g) |
| 6 | 1:0 | 10 | 7 | 0.071 |
| 7 | 4:0:1^{a} | 60 | 7 | 0.076 |
| 8 | 4:1 | 65 | 7 | 0.099 |
| 9 | 3:1 | 70 | 7 | 0.128 |
| 10 | 2:1 | 90 | 20 | 0.138 |
| 11 | 1:1 | 90 | 20 | 0.173 |
| | Ratio of DGS: | | | |
| | Maltonamide-Si(OEt)₃ | Gelation time | Aged | Yield |
| Sample | (w/w) | (min)^{b} | (days) | (g) |
| 12 | 16:0:1^{c} | 55 | 7 | 0.070 |
| 13 | 16:1 | 60 | 7 | 0.081 |
| 14 | 8:1 | 70 | 7 | 0.093 |
| 15 | 4:1 | 70 | 7 | 0.110 |

^{a}Although no trialkoxysilane was present, there was a 4:1 ratio of DGS:sorbitol.
^{b}Refers to the time when the solution ceases to flow after addition of the buffer solution, as judged by repeatedly tilting a test-tube containing the sol until gelation occurred
^{c}Although no trialkoxysilane was present, there was a 16:1 ratio of DGS:Maltose.

TABLE 2
Solid-state ¹³C and ²⁹Si CPMAS NMR
spectral data of samples 8 and 15

| Sample | ¹³C (δ, ppm) | ²⁹Si (δ, ppm) |
|---|---|---|
| 8 | 9.8, 22.8, 41.9, 63.4, 72.7, 174.5 | −66.1, −101.2, −110.5 |
| 15 | 9.3, 22.2, 41.9, 63.4, 72.9, 102.6, 174.8 | −67.2, −101.4, −109.8 |

TABLE 3
Mean Mobilities [25 C, (μ/s)/(V/cm)] of samples 6-11.

| | Sample | | | | | |
|---|---|---|---|---|---|---|
| | 6 | 7 | 8 | 9 | 10 | 11 |
| 1 mM PBS^{a} (pH = 8) | −3.20 | −3.17 | −4.08 | −4.05 | −2.89 | −3.16 |
| 25 mM Tris buffer (pH = 8) | −2.25 | −2.22 | −2.89 | −2.97 | −2.56 | −1.49 |

^{a}PBS = phosphate buffer

TABLE 4
Timing for Gelation (t_{gel}) Phase Separation (tₚₛ)
as a Function of Sol Constituents

| | Gelation | | Phase |
|---|---|---|---|
| | Time | | Separation |
| Gel Materials | (t_{gel}) | | time (tₚₛ) |
| 0.5 g DGS, 500 μL H₂O, 500 μL Tris buffer | 39 | min | never |
| 0.5 g of 0.0259 g/mL PEO (MW 100,000) | 11 | min | 6 min |
| replacing Tris buffer | | | |
| 0.5 g of 0.05 g/mL PEO (MW 100,000) | 7 | min | 5 min |
| replacing Tris buffer | | | |
| 0.5 g MSS,^{a} 800 μL H₂O, 800 μL Tris buffer | >12 | h | never |
| 500 μL PolyNIPAM^{b} solution replacing Tris | 69 | min | 16 min |
| buffer | | | |

^{a}Monosorbitylsilane
^{b}Poly(N-isopropylacrylamide)

TABLE 5
BET results of DGS/PEO gel

| | | |
|---|---|---|
| Surface Area Data (m²/g) | Multi-point BET area | 550 |
| | Langmuir surface area | 1456 |
| Pore Volume Data (cm³/g) | Total pore volume | 0.4585 |
| | | (d < 193.03 nm) |
| Pore Size Data | Average pore | 3.329 |
| | diameters (nm) | |

0.5 g DGS/500 μL H₂O/500 μL PEO solution ( 0.05 g/1000 μL) The sample was crushed, then washed three times with water for 3 days (each time more than 20 mL water). The sample was freeze dried over 24 hours, then degassed at 100 C for 6 hours before measurement.

TABLE 6
Residual PEO in DGS-derived silica

| PEO/ | PEO wt. % | | PEO/Sol | PEO wt. % | | PEO/Sol | PEO wt. % | |
|---|---|---|---|---|---|---|---|---|
| Sol (2K^{a})g/ml | Cal.^{b} | TGA^{c} | (10K^{a})g/ml | Cal. | TGA | (100K^{a})g/ml | Cal. | TGA |
| 0.05 | 24.1 | 25 | 0.025 | 14.2 | 17 | 0.005 | 3.3 | 8 |
| 0.15 | 45.4 | 34 | 0.035 | 20.5 | 23 | 0.015 | 9.1 | 12 |
| 0.25 | 55.0 | 39 | 0.05 | 24.2 | 29 | 0.025 | 14.0 | 19 |
| 0.35 | 60.8 | 23 | 0.15 | 45.2 | 33 | 0.035 | 18.0 | 21 |
| 0.45 | 64.4 | 32 | 0.25 | 54.6 | 34 | 0.05 | 23.6 | 25 |

^{a}Refers to MW of the PEO;
^{b}Calculated based on PEO added;
^{c}Measured using thermogravimetric analysis

TABLE 7
Protein removed by washing silica gel after cure:
Gel entrapped with HSA

| | 1^{st} washing | 2^{nd} washing | 3^{rd} washing |
|---|---|---|---|
| Additive to DGS sol | (μg/mL) | (μg/mL) | (μg/mL) |
| PEO 10K | 77.3 | 54.6 | 7.26 |
| PEO 100K | 98.7 | 4.54 | 0.15 |
| PEO-NH₂ | 117 | 28.3 | 1.34 |
| PEO 10K/PPG-NH₂ 200 | 0 | 0 | 0 |
| PEO 10K/PPG-NH₂ 400 | 0 | 0 | 0 |
| PEO 10K/PAM 17K | 49.4 | 0 | 0 |
| PEO 10K/PAM 65K | 48.2 | 0 | 0 |
| Gluconamide-Si | 0.270 | 0.156 | 0.113 |
| Methyltriethoxysilane | 0.135 | 0.0754 | 0.0704 |
| Phenyltriethoxysilane | 0.428 | 0.0903 | 0.102 |

TABLE 8
Protein removed by washing silica gel after cure:
Gel entrapped with Lysozyme

| | 1^{st} washing | 2^{nd} washing | 3^{rd} washing |
|---|---|---|---|
| Additive to DGS sol | (μg/mL) | (μg/mL) | (μg/mL) |
| PEO 10K | 3.48 | 3.48 | 1.38 |
| PEO 100K | 6.43 | 0.41 | 0.77 |
| PEO-NH₂ | 23.0 | 6.11 | 1.26 |
| PEO 10K/PPG-NH₂ 200 | 0 | 0 | 0 |
| PEO 10K/PPG-NH₂ 400 | 0 | 0 | 0 |
| PEO 10K/PAM 17K | 1.59 | 0.14 | 0 |
| PEO 10K/PAM 65K | 1.17 | 0.16 | 0 |
| Gluconamide-Si | 0.181 | 0.124 | 0.0828 |
| Methyltriexthoxysilane | 0.199 | 0.102 | 0.100 |
| Phenyltriethoxysilane | 0.231 | 0.156 | 0.085 |

TABLE 9
Solutions used to prepare gels from
DGS/PEO and PPG-NH₂

| | PEO (10K^{a}) | | |
|---|---|---|---|
| | 5 g dissolved | 0.5 g PPG-NH₂ 200^{a} | 0.1 g PPG-NH₂ 400^{a} |
| | in 10 mL PBS | (molecular weight)/ | (molecular weight)/ |
| Vial # | (pH8.00, 10 mM)/μl | 1 mL water/μl | 1 mL water/μl |
| 1 | 1000 | 1 | — |
| 2 | 1000 | 5 | — |
| 3 | 1000 | 10 | — |
| 4 | 1000 | — | 10 |
| 5 | 1000 | — | 50 |
| 6 | 1000 | — | 100 |

^{a}refers to molecular weight

TABLE 10
Gels prepared from DGS/PEO and PPG - DGS recipe.

| | Polymer mixture (refer | |
|---|---|---|
| Vial | Table 9 above) μl | Gel time |
| 1 | 60 | ˜60 min |
| 2 | 60 | ˜40 min |
| 3 | 60 | ˜13 min |
| 4 | 60 | ˜42 min |
| 5 | 60 | ˜6 min |
| 6 | 60 | ˜2 min |

TABLE 11
BET data for several silica compositions

| Precursor | | DGS | DGS + PEO2000 | DGS + PEO10K |
|---|---|---|---|---|
| Surface | Single point BET area | 581 | 565 | 560 |
| Area | Multi-point BET area | 596 | 575 | 574 |
| Data | Langmuir surface area | 1668 | 1653 | 1915 |
| (m²/g) | Micro pore area | 473 | 418 | 268 |
| | Meso pore area | 124 | 157 | 305 |
| | Cumulative adsorption | 593 | 503 | 548 |
| | surface area | | | |
| | Cumulative desorption | 586 | 520 | 648 |
| | surface area | | | |
| Pore | Total pore volume | 0.467 | 0.476 | 0.506 |
| Volume | | (<56.2 nm) | (<51.2 nm) | (<54.2 nm) |
| Data | Cumulative adsorption | 0.422 | 0.399 | 0.459 |
| (cm³/g) | pore volume(r = 30-1 nm) | | | |
| | Cumulative desorption | 0.430 | 0.414 | 0.506 |
| | pore volume (r = 30-1 nm) | | | |
| | Micro pore volume | 0.342 | 0.306 | 0.210 |
| Pore | Average pore radius | 1.56 | 1.65 | 1.76 |
| Size | | | | |
| Data | | | | |
| (nm) | | | | |

We claim:

1. A method of preparing siliceous materials comprising combining an organic polyol silane precursor with an additive under conditions suitable for the hydrolysis and condensation of the precursor to a siliceous material, wherein the additive is selected from the group consisting of one or more water-soluble polymers and one or more trifunctional silanes of Formula I: 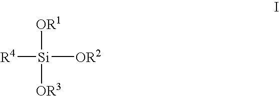
wherein R¹, R² and R³ are the same or different and represent a group that may be hydrolyzed under normal sol-gel conditions to provide Si—OH groups; and R⁴ is group that is not hydrolyzed under normal sol-gel conditions.

2. The method according to claim 1, wherein the additive is a water soluble polymer is selected from the group consisting of polyethers, polyalcohols, polysaccharides, poly(vinyl pyridine), polyacids, polyacrylamides and polyallylamine.

3. The method according to claim 2, wherein the additive is a water soluble polymer is selected from the group consisting of polyethylene oxide (PEO), polyethylene glycol (PEG), amino-terminated polyethylene glycol (PEG-NH₂), polypropylene glycol (PPG), polypropylene oxide (PPO), polypropylene glycol bis(2-amino-propyl ether) (PPG-NH₂), polyvinyl alcohol, poly(acrylic acid), poly(vinyl pyridine), poly(N-isopropylacrylamide) (polyNIPAM) and polyallylamine (PAM).

4. The method according to claim 3, wherein the additive is a water soluble polymer is selected from the group consisting of PEO, PEO—NH₂, PEG, PPG-NH₂, polyNIPAM and PAM.

5. The method according to claim 3, wherein the additive is a water soluble polymer is selected from the group consisting of PEO, PEO—NH₂ and polyNIPAM.

6. The method according to claim 1, wherein the additive is a mixture of water soluble polymers,

7. The method according to claim 6 wherein the mixture of water soluble polymers comprises PEO and PEO—NH₂.

8. The method according to claim 5, wherein the additive is PEO.

9. The method according to claim 8, wherein the PEO has a molecular weight that is greater than about 10,000 g/mol.

10. The method according to claim 9, wherein the PEO is used at a concentration of greater than about 0.005 g/mL of final solution.

11. The method according to claim 5, wherein the additive is PEO—NH₂.

12. The method according to claim 11, wherein the PEO—NH₂ has a molecular weight that is greater than about 3,000 g/mol and is used at a concentration of about 0.005 g/mL of final solution.

13. The method according to claim 5, wherein the water soluble polymer is poly(N-isopropylacrylamide).

14. The method according to claim 13, wherein the poly(N-isopropylacrylamide) has a molecular weight that is about 10,000 g/mol and is used at a concentration of about 0.005 g/mL of final solution.

15. The method according to claim 1 wherein the one ore more additives is a compound of Formula I.

16. The method according to claim 15, wherein OR¹, OR² and OR³ are the same or different and are derived from organic mono-, di-, or polyols.

17. The method according to claim 16, wherein OR¹, OR² and OR³ are the same or different and are derived from sugar alcohols, sugar acids, saccharides, oligosaccharides and polysaccharides.

18. The method according to claim 17, wherein OR¹, OR² and OR³ are the same or different and are derived from allose, altrose, glucose, mannose, gulose, idose, galactose, talose, ribose, arabinose, xylose, lyxose, threose, erythrose, glyceraldehydes, sorbose, fructose, dextrose, levulose, sorbitol, sucrose, maltose, cellobiose, lactose, dextran, (500-50,000 MW), amylose, pectin, glycerol, propylene glycol and trimethylene glycol.

19. The method according to claim 18, wherein OR¹, OR² and OR³ are the same or different and are derived from glycerol, sorbitol, maltose, trehalose, glucose, sucrose, amylose, pectin, lactose, fructose, dextrose and dextran.

20. The method according to claim 18, wherein OR¹, OR² and OR³ are the same or different and are derived from glycerol, sorbitol, maltose and dextran.

21. The method according to claim 15, wherein OR¹, OR² and OR³ are the same or different and are selected from C₁₋₄alkoxy, aryloxy and arylalkyleneoxy.

22. The method according to claim 21, wherein wherein OR¹, OR² and OR³ are the same or different and are selected from C₁₋₄alkoxy, phenyoxy, naphthyloxy and benzyloxy.

23. The method according to claim 22, wherein wherein OR¹, OR² and OR³ are the same or different and are selected from C₁₋₄alkoxy.

24. The method according to claim 23, wherein OR¹, OR² and OR³ are all ethoxy.

25. The method according to claim 15, wherein R⁴ is selected from one of the following groups:
polyol-(linker)-;
polymer-(linker)ₙ-; and 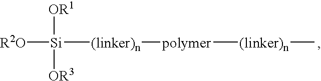
wherein n is 0-1.

26. The method according to claim 25, wherein the polyol is an organic mono-, di-, or polyol.

27. The method according to claim 26, wherein the polyol is selected from the group consisting of a sugar alcohol, sugar acid, saccharide, oligosaccharide and polysaccharide.

28. The method according to claim 27, wherein the polyol is a selected from the group consisting of allose, altrose, glucose, mannose, gulose, idose, galactose, talose, ribose, arabinose, xylose, lyxose, threose, erythrose, glyceraldehydes, sorbose, fructose, dextrose, levulose, sorbitol, sucrose, maltose, cellobiose, lactose, dextran, (500-50,000 MW), amylose, pectin, glycerol, propylene glycol and trimethylene glycol.

29. The method according to claim 28, wherein the polyolos a selected from the group consisting of glycerol, sorbitol, maltose, trehalose, glucose, sucrose, amylose, pectin, lactose, fructose, dextrose and dextran.

30. The method according to claim 29, wherein he polyol is selected from the group consisting of glycerol, sorbitol, maltose and dextrose.

31. The method according to claim 25 wherein the polymer is a water soluble polymer.

32. The method according to claim 31, wherein the polymer is selected from the group consisting of polyethylene oxide (PEO), polyethylene glycol (PEG), amino-terminated polyethylene glycol (PEG-NH₂), polypropylene glycol (PPG), polypropylene oxide (PPO), polypropylene glycol bis(2-amino-propyl ether) (PPG-NH₂), polyvinyl alcohol, poly(acrylic acid), poly(vinyl pyridine), poly(N-isopropylacrylamide) (polyNIPAM) and polyallylamine (PAM).

33. The method according to claim 32, wherein the water soluble polymer is selected from the group consisting of PEO, PEO—NH₂, PEG, PPG-NH₂, polyNIPAM and PAM.

34. The method according to claim 33, wherein the polymer is PEO.

35. The method according to claim 25, wherein the linker is selected from the group consisting of C₁₋₂₀alkylene, C₁₋₂₀alkenylene, organic ethers, thioethers, amines, esters, amides, urethanes, carbonates and ureas.

36. The method according to claim 25, wherein the compound of Formula I is selected from the group consisting of:
GluconamideSi (Compound 1);
MaltonamideSi (Compound 2);
DextronamideSi (Compound 3);
(CH₂CH₂O)ₚ[(EtO)₃Si(C₃H₆)]₂, p˜4-5, average MW 200 (Compound 5a);
(CH₂CH₂O)ₚ[(EtO)₃Si(C₃H₆)]₂, p˜13, average MW 600 (Compound 5b);
(CH₂CH₂O)ₚ[(EtO)₃Si(C₃H₆)]₂, p˜44, average MW 2000 (Compound 5c); and
(CH₂CH₂O)ₚ[(EtO)₃Si(C₃H₆)]₂, p˜227, average MW 10,000 (Compound 5d).

37. The method according to claim 1, wherein the organic polyol silane precursor is selected from the group consisting of diglycerylsilane (DGS), monosorbitylsilane (MSS), monomaltosylsilane (MMS), dimaltosylsilane (DMS) and a dextran-based silane (DS).

38. The method according to claim 1, wherein the conditions suitable for the hydrolysis and condensation of the precursor to a siliceous material include a pH in the range of about 4-11.5 in aqueous solutions and with optional sonication to assist in dissolution.

39. A method of preparing siliceous materials with low shrinkage characteristics comprising:
(a) combining an aqueous solution of one or more compounds of Formula I: 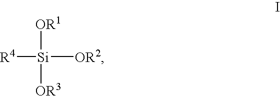
wherein R¹, R² and R³ are the same or different and represent a group that may be hydrolyzed under normal sol-gel conditions to provide Si—OH groups; and R⁴ is group that is not hydrolyzed under normal sol-gel conditions with an aqueous solution of an organic polyol silane precursor;
(b) adjusting the pH of the solution in (a) to about 4-11.5;
(c) allowing the solution of (b) to gel;
(d) aging the gel of (c); and
(e) drying the aged gel in air.

40. A siliceous material prepared using the method according to claim 1.

41. A method of preparing monolithic silica materials comprising combining an organic polyol silane precursor with one or more additives selected from water-soluble polymers and compounds of Formula I: 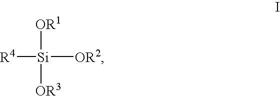
wherein R⁴ is group selected from polymer-(linker)ₙ- and 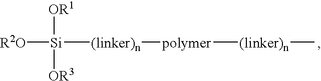
under conditions where a phase transition occurs before gelation.

42. The method according to claim 41, wherein R⁴ is

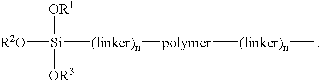

43. The method according to claim 42, wherein the linker group is a C₁₋₄alkylene group and n is 1.

44. The method according to claim 42, wherein OR¹, OR² and OR³ are the same and are selected from C₁₋₄alkoxy.

45. The method according to claim 42, wherein the polymer is PEO.

46. The method according to claim 41 wherein the compound of Formula I is selected from the group consisting of:
(CH₂CH₂O)ₚ[(EtO)₃Si(C₃H₆)]₂, p˜4-5, average MW 200 (Compound 5a);
(CH₂CH₂O)ₚ[(EtO)₃Si(C₃H₆)]₂, p˜13, average MW 600 (Compound 5b);
(CH₂CH₂O)ₚ[(EtO)₃Si(C₃H₆)]₂, p˜44, average MW 2000 (Compound 5c); and
(CH₂CH₂O)ₚ[(EtO)₃Si(C₃H₆)]₂, p˜227, average MW 10,000 (Compound 5d).

47. The method according to claim 41, wherein the water soluble polymer is selected from the group consisting of PEO, PEO—NH₂ and poly(NIPAM).

48. A bimodal meso/macroporous silica monolith prepared using the method according to claim 41.

49. A method of preparing siliceous materials comprising combining an organic polyol silane precursor, a biomolecule of interest and an additive under conditions suitable for the hydrolysis and condensation of the precursor to a siliceous material, wherein the additive is selected from the group consisting of one or more water-soluble polymers and one or more trifunctional silanes of Formula I: 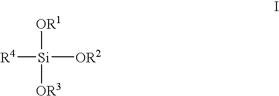
wherein R¹, R² and R³ are the same or different and represent a group that may be hydrolyzed under normal sol-gel conditions to provide a Si—OH group; and R⁴ is group that is not hydrolyzed under normal sol-gel conditions.

50. A siliceous material comprising a biomolecule entrapped therein wherein the siliceous material is prepared using the methods according to claim 49.

51. A method for the quantitative or qualitative detection of a test substance that reacts with or whose reaction is catalyzed by an active biological substance, wherein said biological substance is encapsulated within a siliceous material, comprising:
(a) preparing the siliceous material comprising said active biological substance entrapped within a porous, silica matrix using a method according to claim 49;
(b) bringing said biological-substance-containing siliceous material into contact with a gas or aqueous solution comprising the test substance; and
(c) quantitatively or qualitatively detecting, observing or measuring the change in one or more characteristics in the biological substance entrapped within the siliceous material or, alternatively, quantitatively or qualitatively detecting, observing or measuring the change in one or more characteristics in the test substance.

52. The method according to claim 51, wherein the change in one or more characteristics of the entrapped biological substance is qualitatively or quantitatively measured by spectroscopy, utilizing one or more techniques selected from the group consisting of UV, IR, visible light, fluorescence, luminescence, absorption, emission, excitation and reflection.

53. A method of storing a biologically active biological substance in a silica matrix, wherein the biological substance is an active protein or active protein fragment, wherein the silica matrix prepared using a method according to claim 49.

54. A method of preparing a monolithic silica chromatographic column comprising placing a solution comprising an organic polyol silane precursor and one or more additives selected from water-soluble polymers and a compound of Formula I: 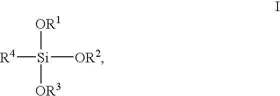
wherein R⁴ is group selected from polymer-(linker)ₙ- and 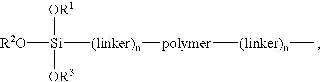
in a column under conditions suitable for a phase transition to occur before gelation.

55. The method according to claim 54, wherein the solution further comprises one or more substances, which provide cationic sites that counterbalance the anionic charge of the silica to reduce non-selective interactions

56. A chromatographic column comprising a silica monolith prepared by combining an organic polyol silane precursor and one or more additives selected from water-soluble polymers and a compound of Formula 1: 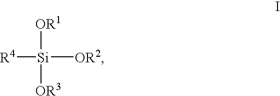
wherein R⁴ is group selected from polymer-(linker)ₙ- and 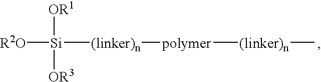
under conditions where a phase transition occurs before gelation.

57. A method of preparing a monolithic silica column having an active biomolecule entrapped therein comprising combining:
a) a polyol-silane derived silica precursor;
b) one or more additives selected from water soluble polymers and a compound of Formula I: 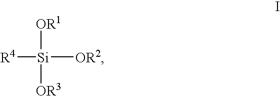
wherein R⁴ is group selected from polymer-(linker)ₙ- and 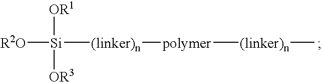
and
c) a biomolecule;
under conditions wherein a phase separation occurs before gelation.

58. The method according to claim 57, wherein the additive is one or more water soluble polymers or a compound of Formula I, wherein R⁴ is

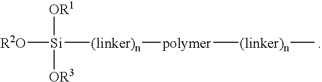

59. The method according to claim 57, wherein the organic polyol silane silica precursor, one or more additives and biomolecule are also combined with a substance which provides cationic sites that counterbalance the anionic charge of the silica to reduce non-selective interactions, for example, aminopropyltriethoxysilane (APTES), PAM, PPG-NH₂ and PEG-NH₂.

60. A chromatographic column prepared using a method according to claim 57.

61. A use of the column according to claim 59, in methods for immunoaffinity chromatography, sample cleanup, solid phase extraction or preconcentration of analytes, removal of unwanted contaminants (for example by antibody binding), solid phase catalysis and frontal affinity chromatography (with or without mass spectral detection).
